(19) United States
(12) Patent Application Publication
(10) Pub. No.: US 2020/0206137 A1
(43) Pub. Date: Jul. 2, 2020

(54) MICROPARTICLE FORMULATIONS FOR DELIVERY OF ACTIVE AGENTS

(71) Applicant: WOLFCREEK BIOTECH PTE LTD, SINGAPORE (SG)

(72) Inventors: Tjhang Jessica Gambino, Singapore (SG); Cherry Chooi Ling KHOO, Singapore (SG)

(73) Assignee: WOLFCREEK BIOTECH PTE LTD, SINGAPORE (SG)

(21) Appl. No.: 16/631,436

(22) PCT Filed: Jul. 17, 2018
(86) PCT No.: PCT/IB2018/055266

§ 371 Date: Jan. 15, 2020

Related U.S. Application Data
(60) Provisional application No. 62/533,534, filed on Jul. 17, 2017.
(60) Provisional application No. 62/533,537, filed on Jul. 17, 2017.

(51) Int. Cl. A61K 9/16; A61K 9/50; A61K 9/00; A61K 31/5575; A61P 27/06

(52) U.S. Cl. CPC A61K 9/1647; A61K 9/5026; A61K 9/1635; A61K 31/5575; A61P 27/06; A61K 9/0048

(57) ABSTRACT

Provided herein are polymer microparticle-based compositions for the treatment of ocular diseases/disorders (e.g., glaucoma) and other diseases/disorders. Microparticle suspension formulations and solid polymer formulations are described, which provide extended ocular residence time and controlled release of therapeutic agents such as latanoprost, atropine, brimonidine, timolol, brinzolamide, dorzolamide, octyl methoxycinnamate (OMC) and benzophenone-3 (BP3). In specific embodiments, a topical composition comprising drug loaded poly(lactic-co-glycolic acid (PLGA) microparticles or chitosan-coated drug-loaded PLGA microparticles is prepared.

### CROSS-REFERENCES TO RELATED APPLICATIONS

The present application claims priority to U.S. Provisional Pat. Appl. No. 62/533,534, filed Jul. 17, 2017, and U.S. Provisional Pat. Appl. No. 62/533,537, filed Jul. 17, 2017, which applications are incorporated herein by reference in their entirety.

### BACKGROUND OF THE INVENTION

Current drug delivery systems such as topical eye drops are rapidly washed off from the surface of the eye within a few minutes of administering the drug and only about 5% of the eye-drop actually reaches the eye tissues. The low bioavailability and rapid clearance of drugs from the ocular surface, has led to high frequency of dosage administration. Patient compliance with dosage regimens and side effects of topical medications have prevented successful treatment of eye diseases such as glaucoma. Several studies have shown that about 50% of glaucoma patients have not been adherent to their medication over 75% of the time.

In addition to poor compliance on part of glaucoma patients, the ageing population worldwide also drives an increasing demand for a sustained eye-drug delivery system.

Most eye drops for ocular treatment simply contain various drug molecules in solution, and suffer from rapid clearance from the eye in a few seconds, thus necessitating repeated application (2-4 times daily in most cases) (Ali et al. Advanced Drug Delivery Reviews. 2006, 58: 1258-1268). The use of “carriers” for sustained drug delivery typically involves administration of aqueous biodegradable gels to the eye, which release drug molecules slowly over a longer period. Ophthalmic gel forming solutions are isotonic, buffered aqueous solution which can contain timolol maleate, the active ingredient in reducing elevated intraocular pressure in normal or glaucomatous eyes. On contact with the pre-corneal tear film, the solution gels and is subsequently removed by flow of tears. A single dose of the solution provides a 12-hour reduction in intraocular pressure.

Also known are eye drops in the form of sterile ophthalmic resin suspensions in aqueous solution. Such eye drops can contain betaxolol hydrochloride which reduces elevated intraocular pressure in normal or glaucomatous eyes. A single dose provides a 12-hour reduction in intraocular pressure. The above formulations contain viscosifying polymers such as gellan gum and carbomers to increase the bioavailability of the drug. However none of these formulations are able to achieve a more sustained drug delivery to reduce the frequency of ocular drug administration.

Methods and systems for longer and more sustained ocular drug delivery typically involve invasive techniques. Drug-eluting intracanalicular plugs have to be implanted to deliver intended drugs. While there is more sustained drug delivery, the implantation of such plugs present a high risk to patient and there is also a risk of plug dislodgement. Injectable, bioerodible micro-inserts are currently being developed which can provide up to a month of sustained drug delivery. However these micro-inserts present a high risk to patients because there is only one chance for proper administration; injection at an incorrect location will render the patient visit wasted, and the patient will have to revert to eye drops. In addition, the invasive systems described above require professional medical training, can be costly and will likely inconvenience patients.

There is accordingly a need for improved formulations for sustained ocular drug delivery, which are easy to use and simple to administer. There is also a need for an ocular drug formulation which can combine multiple dosage regimes into a single action for improved convenience to patients. The present invention addresses this and other needs.

### SUMMARY OF THE INVENTION

The present disclosure provides polymer microparticle-based compositions for the treatment of ocular diseases/disorders (e.g., glaucoma) and other diseases/disorders. The compositions can be tuned in terms of polymer composition, polymer molecular weight, particle size, cargo loading levels, and surface properties to provide advantages including, but not limited to, exceptionally long residence times in the eyes of subjects to whom the compositions are administered. The combination of extended residence time and controlled cargo release can provide round-the-clock therapeutic benefits, improve patient compliance, and reduce complications associated with traditional treatment regimens such as eye drops.

Exemplary embodiments provided in accordance with the presently disclosed subject matter include, but are not limited to, the claims and the following embodiments:
1. A composition comprising a population of polymer particles comprising a cargo, wherein the particles have an average particle size ranging from about 1 μm to about 25 μm.
2. The composition of embodiment 1, wherein the particles are adapted to carry and release the cargo upon topical ocular administration to a subject.
3. The composition of embodiment 1 or embodiment 2, wherein the polymer is selected from the group consisting of poly(lactic-co-glycolic acid), polylactic acid, poly(glycolic acid), poly(acrylic acid), alginate, a poly(alkyl cyanoacrylate), cellulose acetate phthalate, poly(ethyl cyanoacrylate), poly(hexadecyl cyanoacrylate), polycaprolactone, polylactic acid-polyethylene glycol copolymer, poly(lactic-co-glycolic acid)-polyethylene glycol copolymer, and combinations thereof.
4. The composition of embodiment 3, wherein the polymer is poly(lactic-co-glycolic acid).
5. The composition of embodiment 4, wherein the poly(lactic-co-glycolic acid) has a molecular weight ranging from about 4 kDa to about 150 kDa (weight average).
6. The composition of embodiment 4 or embodiment 5, wherein the molecular weight ranges from about 7 kDa to about 17 kDa (weight average).
7. The composition of any one of embodiments 4-6, wherein the molar ratio of lactic acid to glycolic acid in the poly(lactic-co-glycolic acid) ranges from about 5:95 to about 95:5.
8. The composition of embodiment 7, wherein the molar ratio of lactic acid to glycolic acid is about 50:50.
9. The composition of any one of embodiments 1-8, wherein the average particle size ranges from about 10 μm to about 20 μm.
10. The composition of any one of embodiments 1-9, wherein the cargo comprises one or more ophthalmic therapeutic agents.
11. The composition of embodiment 10, wherein the cargo comprises two or more ophthalmic therapeutic agents.
12. The composition of any one of embodiments 1-9, wherein the cargo comprises a prostaglandin, a carbonic anhydrase inhibitor, an alpha agonist, a beta blocker, a UV blocker, or a combination thereof
13. The composition of any one of embodiments 1-9, wherein the cargo comprises latanoprost.
14. The composition of any one of embodiments 1-13, wherein the amount of cargo ranges from about 0.1% (w/w) to about 50% (w/w) based on the total weight of the particles.
15. The composition of embodiment 14, wherein the amount of cargo ranges from 1% (w/w) to about 20% (w/w) based on the total weight of the particles.
16. The composition of any one of embodiments 1-15, wherein the cargo comprises a further population of particles, the further population of particles comprising at least one drug.
17. The composition of any one of embodiments 1-16, wherein the particles are coated with a mucoadhesive coating.
18. The composition of embodiment 17, wherein the mucoadhesive coating comprises chitosan.
19. The composition of embodiment 1, wherein:
the average particle size ranges from about 10 μm to about 20 μm;
the polymer is poly(lactic-co-glycolic acid) having a molecular weight ranging from about 7 kDa to about 17 kDa (weight average), wherein the molar ratio of lactic acid to glycolic acid in the poly(lactic-co-glycolic acid) is about 50:50;
the cargo comprises a prostaglandin, a carbonic anhydrase inhibitor, an alpha agonist, a beta blocker, a UV blocker, or a combination thereof;
the amount of cargo ranges from 1% (w/w) to about 20% (w/w) based on the total weight of the particles; and
the particles are coated with a mucoadhesive polymer comprising chitosan.
20. The composition of any one of embodiments 1-19, wherein the particles are suspended in a fluid medium.
21. The composition of any one of embodiments 1-19, wherein the particles are partially or fully embedded in a solid polymer matrix.
22. The composition of embodiment 21, wherein the solid polymer matrix comprises one or more polymers selected from the group consisting of polyvinyl alcohol, poly(ethylene glycol), polyvinyl pyrrolidone, polyacrylic acid, polyacrylamide, poly(N-2-hydroxypropyl) methylacrylamide), poly(methyl vinyl ether-alt-maleic anhydride), and a poly(2-alkyl-2-oxazoline).
23. The composition of embodiment 21 or embodiment 22, wherein the solid polymer matrix comprises polyvinyl alcohol.
24. The composition of any one of embodiments 1-23, which is formulated as an ophthalmic composition for administration to an eye of the subject.
25. A composition according to any one of embodiments 1-24 for use in the treatment of an ocular disease or disorder in a patient.
26. The composition of embodiment 25, wherein the ocular disease or disorder is glaucoma.
27. A composition according to any one of claims 1-24 for use in the manufacture of a medicament for the treatment of an ocular disease or disorder.
28. The composition of embodiment 27, wherein the ocular disease or disorder is glaucoma.
29. A method for treating glaucoma, the method comprising administering an effective amount of a composition according to any one of embodiments 1-24 to a subject in need thereof.
30. A kit comprising a first container comprising a composition according to any one of embodiments 1-20 and a second container comprising a fluid medium for suspension of the particles in the composition, wherein the fluid medium optionally comprises one or more pharmaceutically acceptable excipients.
31. The kit of embodiment 30, wherein the fluid medium is aqueous.
32. The kit of embodiment 30 or embodiment 31, wherein the fluid medium comprises dissolved cargo.
33. The kit of any one of embodiments 30-32, wherein the concentration of the dissolved cargo is at or around the solubility limit of the cargo in the fluid medium.
34. The kit of embodiment 30, further comprising instructions for suspending the particles in the fluid medium.

### BRIEF DESCRIPTION OF THE DRAWINGS

FIG. 1 shows a schematic experimental setup for generation of emulsion drops using capillary microfluidics, followed by solvent evaporation of formulated droplets in a glass well to form polymeric PLGA particles.

FIG. 2A shows a microscopic image of fabricated poly(lactic-co-glycolic acid) (PLGA) particles co-formulated with latanoprost.

FIG. 2B shows the size distribution histogram of fabricated microparticles. The mean diameter was 15 μM with a standard deviation of 5%.

FIG. 2C shows a microscopic image of fabricated poly(lactic-co-glycolic acid) (PLGA) particles co-formulated with red fluorescent dye, Nile red. Visualization using blue light and amber filter. Magnification at 4×.

FIG. 3 shows the release profile of latanoprost over a period of 7 days from PLGA particles, with slow sustained release.

FIG. 4 shows images of rabbit eye (lacrimal laruncle, 0.63×) following administration of dye-loaded microparticles.

FIG. 5 shows an image of rabbit eye (lower fornix, 0.63×) at day 7 following administration of dye- and latanoprost-loaded microparticles.

FIG. 6 shows the effects of latanoprost-loaded PLGA microparticles on intraocular pressure following a single dose in dogs, as compared to latanoprost eye drops (XALATAN). Data represent the mean±S.E.M of 5 eyes. * <0.05, ** p<0.01 vs. vehicle by Student's t-test.

FIG. 7A shows the drug release profile of atropine-loaded PLGA microparticles in PBS at 37° C. Standard deviations obtained from 2 replicates, n=1 for sample at 7 days.

FIG. 7B shows the drug release profile of brimonidine-loaded PLGA microparticles in PBS at 37° C. Standard deviations obtained from 3 replicates.

FIG. 7C shows the drug release profile of timolol-loaded PLGA microparticles in PBS at 37° C.

FIG. 8A shows the drug release profile of brinzolamide-loaded PLGA microparticles in PBS at 37° C.

FIG. 8B shows the drug release profile of dorzolamide-loaded PLGA microparticles in PBS at 37° C.

FIG. 9A shows the release of octyl methoxycinnamate (OMC) from PLGA microparticles in PBS at 37° C.

FIG. 9B shows the release of benzophenone-3 (BP-3) from PLGA microparticles in PBS at 37° C.

FIG. 10 shows in vitro latanoprost release profile from PLGA microparticles with different LA:GA ratios, molecular weights and particle size. Error bars represent standard deviations from n=3 [(75:25) Mw 66-107 kDa, 180 μm], n=2 [(50:50) Mw 30-60 kDa, 17 μm] and [(50:50) Mw 7-17 kDa, 17 μm].

### DETAILED DESCRIPTION OF THE INVENTION

### I. Definitions

Unless otherwise stated, the term “average” is synonymous with “mean” in the specification herein and has an ordinary meaning in the art. Further, unless otherwise stated, “particle size” and “particle diameter” are synonymous in the specification herein and can be measured by methods known in the art, which include but are not limited to light-scattering methods and microscopy.

As used herein, an “individual” refers to human and animal subjects. Further, as used herein, a “patient” refers to a subject afflicted with a disease and/or disorder, and includes human and animal subjects. Furthermore, the terms “treatment” and “treat” and synonyms thereof used herein, refer to both ophthalmic therapeutic treatment and prophylactic or preventative measures, wherein the object is to cure, prevent, or slow down (lessen) the condition of a disease and/or disorder, such as an ocular disease and/or disorder.

As used herein, the term “mucoadhesive agent” refers to any agent which exhibits an affinity for the surface of a mucous membrane (e.g., the ocular mucosa), thereby promoting adherence to the surface. Adherence to the surface generally occurs via non-covalent interactions including hydrogen bonding and van der Waals forces, which can with the mucous or the underlying cells. Examples of mucoadhesive agents include, but are not limited to, poloxamers, carbomers, hyaluronan, and chitosan. The use of coated particles ranging in size from about 1 μm to about 25 μm has been discovered to be particularly advantageous for topical delivery of active agents to the eye with extended ocular residence times.

As used herein, the term “ophthalmic therapeutic agent” refers to a drug used for treating a disease or condition affecting the eye.

As used herein, the term “latanoprost” refers to (5Z)-7-[(1R,2R,3R,5S)-3,5-dihydroxy-2-[(3R)-3-hydroxy-5-phenylpentyl]cyclopentyl]-5-heptenoic acid 1-methylethyl ester (CAS Registry No. 130209-82-4) and pharmaceutically acceptable salts thereof.

As used herein, the term “dexamethasone” refers to (11β,16α)-9-fluoro-11,17,21-trihydroxy-16-methyl-pregna-1,4-diene-3,20-dione (CAS. Registry No. 50-02-2) and pharmaceutically acceptable salts thereof

As used herein, the terms “poly(lactic acid-co-glycolic acid),” “poly(lactide-co-glycolide,” “PLGA,” and variants thereof refer to any copolymer—including block copolymers and random copolymers—containing lactic acid monomers and glycolic acid monomers covalently bonded via ester bonds. PLGA polymers can vary in molecular weight and size distribution (i.e., polydispersity), and all such polymers are contemplated for use in the compositions and methods of the invention.

As used herein, the terms “about” and “around” indicate a close range around a numerical value when used to modify that specific value. If “X” were the value, for example, “about X” or “around X” would indicate a value from 0.9X to 1.1X, e.g., a value from 0.95X to 1.05X, or a value from 0.98X to 1.02X, or a value from 0.99X to 1.01X. Any reference to “about X” or “around X” specifically indicates at least the values X, 0.9X, 0.91X, 0.92X, 0.93X, 0.94X, 0.95X, 0.96X, 0.97X, 0.98X, 0.99X, 1.01X, 1.02X, 1.03X, 1.04X, 1.05X, 1.06X, 1.07X, 1.08X, 1.09X, and 1.1X, and values within this range.

### II. Microparticles

Particles used in the compositions and methods of the invention are capable of carrying and releasing a cargo. In some embodiments, the particles are adapted to release the cargo at a controlled rate, which can contribute to the sustained release of the cargo being delivered to the intended cells and/or tissues. The release of the cargo from the particles can occur at controlled, sustained rates over a period of time (e.g., a 5-day period). As used throughout the specification, the term “release” means to make something available, and said term includes elution. The term “cargo” used throughout the specification refers to something that the particles in the present invention are adapted to carry and release, and the term includes, but is not limited to drugs and other particles having smaller average particles sizes, e.g., nanoparticles. Preferred cargoes include drugs, e.g., ophthalmic therapeutic agents. Depending on the application, the cargo may be carried within each particle or on a surface of each particle.

A formulation/composition capable of sustained release will be understood to refer to a formulation/composition which is capable of releasing its cargo(s) over a time period longer than what is known in the art, particularly in comparison with a gold standard, for example, if a known formulation is capable of releasing a drug over a 12 hour period, a sustained release of that same drug by the formulation of the present invention would be more than a 12 hour period, e.g., a 24 hour period, a 5 day period, or a one month period. In some embodiments, a formulation/composition capable of sustained release refers to a formulation capable of releasing its cargo(s) over a period of 5 or more days. The rate of release of the particles' cargos will depend on the application and can be varied by changing, for example, the particle size and/or the porosity of the material of the particles.

The present invention also relates to an pharmaceutical composition (as described above) for use in the treatment of an ocular disease and/or disorder. In some embodiments, the ocular disease or disorder is glaucoma. Glaucoma is an eye condition characterized by optic nerve damage and is defined as high intraocular pressure (TOP) in the eye, caused due to the imbalance between fluid production and fluid drainage in the eye. This disease worldwide is set to increase from 25 million to 76 million by 2020 and to 111.8 million by 2040 (Quigley, et al. British journal of ophthalmology, 2006, 90(3): 262-267). Current treatment methods for glaucoma consist essentially of drugs, laser treatment, and surgery. The most common non-surgical treatment for regulating TOP levels in the eye is the topical administration of drugs on the surface of the eye (using eye drops and like formulations). 90% of glaucoma patients treated medically are treated with prostaglandins or analogs thereof, e.g., latanoprost (marketed as XALATAN) or bimatoprost (marketed as LUMIGAN).

The present invention also relates to an composition for use in the manufacture of a medicament for the treatment of an ocular disease and/or disorder. In some embodiments, the ocular disease or disorder is glaucoma.

The present invention also relates to a method of treating an ocular disease and/or disorder. In some embodiments, the ocular disease or disorder is glaucoma. The method involves administering to a patient, a therapeutically effective amount of the pharmaceutical composition (as described above) of the present invention.

In certain embodiments, the average size of the particles ranges from about 1 μm to about 100 μm. The particle size can range, for example, from about 1 μm to about 5 μm, or from about 5 μm to about 10 μm, or from about 10 μm to about 20 μm, or from about 20 μm to about 30 μm, or from about 30 μm to about 40 μm, or from about 40 μm to about 50 μm, or from about 50 μm to about 60 μm, or from about 60 μm to about 70 μm, or from about 70 μm to about 80 μm, or from about 80 μm to about 90 μm, or from about 90 μm to about 100 μm. The particle size can range, for example, from about 13 μm to about 18 μm, or from about 10 μm to about 25 μm, or from about 5 μm to about 30 μm. In certain embodiments, the average size of at least one of the populations of particles is around 1, 2, 3, 4, 5, 6, 7, 8, 9, 10, 11, 12, 13, 14, 15, 16, 17, 18, 19, 20, 21, 22, 23, 24, or 25 μm. In some embodiments, the average size of at least one of the populations of particles is less than 150 μm. In some embodiments, the average size of at least one of the populations of particles is less than 145 μm.

Particle sizes ranging from 1 to 25 μm can be particularly advantageous, because they have been found to reduce the foreign body sensation in the eye following ocular administration relative to larger particles which, in turn, can reduce tear drainage of the composition by reducing the stimulation of the reflex arc of the fifth and seventh cranial nerves. In addition, particle sizes below 1 μm can contribute to accumulation of particles and particle cargo in off-target tissues.

In some embodiments, the particles are microparticles. In some embodiments, the average particle size ranges from 1 μm to 25 μm. The actual particle size of each particle need not be exactly the same, so long as the average particle size of all of these particles fall within the intended size ranges.

The particles of the present invention are precision fabricated and are made of a biocompatible matrix material which exhibits sustained release of their cargos, e.g., drugs over a period of time. In some embodiments, the biocompatible material is a polymer. Such biocompatible material may be biodegradable or non-biodegradable. Such biocompatible material includes but is not limited to polylactic acid (PLA), poly(glycolic acid) (PGA), poly(lactic-co-glycolic acid) (PLGA), poly(acrylic acid) (PAA), alginate, a poly(alkyl cyanoacrylate) such as poly(butyl cyanoacrylate) or poly(isobutyl cyanoacrylate), cellulose acetate phthalate, poly(ethyl cyanoacrylate), poly(hexadecyl cyanoacrylate), polycaprolactone, polylactic acid-polyethylene glycol copolymer, poly(lactic-co-glycolic acid)-polyethylene glycol copolymer, and combinations thereof. In some embodiments, the biocompatible material is selected from PLA, PGA, PLGA, PAA, alginate, a poly(alkyl cyanoacrylate) such as poly(butyl cyanoacrylate) or poly(isobutyl cyanoacrylate), cellulose acetate phthalate, poly (ethyl cyanoacrylate), and poly (hexadecyl cyanoacrylate).

In certain embodiments, the particles contain PLGA. In some embodiments, the particles consist essentially of PLGA and cargo material(s). The molecular weight of the PLGA can be varied to control properties such as cargo loading capacity, the rate of cargo release, and the size of the particles. PLGA polymers can be used with molecular weights (weight average or number average) ranging from 4 kDa to 150 kDa, e.g., 66 kDa to 107 kDa. The molecular weight can range from about 4 kDa to about 10 kDa (weight average), or from about 10 kDa to about 25 kDa (weight average), or from about 25 kDa to about 50 kDa (weight average), or from about 50 kDa to about 75 kDa (weight average), or from about 75 kDa to about 100 kDa (weight average), or from about 100 kDa to about 125 kDa (weight average), or from about 125 kDa to about 150 kDa (weight average). The molecular weight can range from about 60 kDa to about 70 kDa (weight average), or from about 50 kDa to about 80 kDa (weight average), or from about 40 kDa to about 90 kDa (weight average), or from about 30 kDa to about 100 kDa (weight average), or from about 20 kDa to about 110 kDa (weight average), or from about 10 kDa to about 120 kDa (weight average), or from about 5 kDa to about 130 kDa (weight average), or from about 4 kDa to about 140 kDa (weight average).

The ratio of lactic acid to glycolic acid in the PLGA can also be varied to control drug loading capacity and other properties. The molar ratio of lactic acid to glycolic acid in the PLGA can range for example, from about 5:95 to about 95:5, or from about 10:90 to about 90:10, or from about 20:80 to about 80:20, or from about 30:70 to about 70:30, or from about 40:60 to about 60:40. The molar ratio of lactic acid to glycolic acid in the PLGA can range from about 45:55 to about 55:45, or from about 40:60 to about 55:45, or from about 35:85 to about 55:45, or from about 30:70 to about 55:45, from about 45:55 to about 60:40, or from about 35:85 to about 60:40, or from about 30:70 to about 60:40. In some embodiments, the ratio of the lactic acid to glycol acid in the PLGA is about 50:50.

In some embodiments, the ratio of the lactic acid to glycol acid in the PLGA is about 50:50 and the molecular weight of the PLGA ranges from about 4 kDa to about 10 kDa (weight average), or from about 10 kDa to about 25 kDa (weight average), or from about 25 kDa to about 50 kDa (weight average), or from about 50 kDa to about 75 kDa (weight average), or from about 75 kDa to about 100 kDa (weight average), or from about 100 kDa to about 125 kDa (weight average), or from about 125 kDa to about 150 kDa (weight average). In some embodiments, the ratio of the lactic acid to glycol acid in the PLGA is about 50:50 and the molecular weight of the PLGA ranges from about 5 kDa to about 20 kDa, e.g., 7-17 kDa (weight average). In some embodiments, the ratio of the lactic acid to glycol acid in the PLGA is about 50:50 and the molecular weight of the PLGA ranges from about 20 kDa to about 60 kDa, e.g., 30-60 kDa, 20-40 kDa, or 24-38 kDa (weight average).

In some embodiments, the ratio of the lactic acid to glycol acid in the PLGA is about 50:50 and the molecular weight of the PLGA ranges from about 60 kDa to about 70 kDa (weight average), or from about 50 kDa to about 80 kDa (weight average), or from about 40 kDa to about 90 kDa (weight average), or from about 30 kDa to about 100 kDa (weight average), or from about 20 kDa to about 110 kDa (weight average), or from about 10 kDa to about 120 kDa (weight average), or from about 5 kDa to about 130 kDa (weight average), or from about 4 kDa to about 140 kDa (weight average).

Non-degradable polymers useful in the preparation of the microspheres include polyethers, vinyl polymers, polyurethanes, cellulose-based polymers, and polysiloxanes. Exemplary polyethers include poly (ethylene oxide), poly (ethylene glycol), and poly (tetramethylene oxide). Exemplary vinyl polymers include polyacrylates, acrylic acids, poly (vinyl alcohol), poly (vinyl pyrrolidone), and poly (vinyl acetate). Exemplary cellulose-based polymers include cellulose, alkyl cellulose, hydroxyalkyl cellulose, cellulose ethers, cellulose esters, nitrocellulose, and cellulose acetates. Depending on the application, the particles in the present invention may be fabricated from one or more different types of biocompatible materials.

The particles in the present invention are capable of carrying cargos, preferably drugs. Hydrophobic drugs are particularly preferred. As used herein in reference to the drug, the term “hydrophobic” refers to a bioactive agent that has solubility in water of no more than 10 milligrams per milliliter (10 mg/mL). In some embodiments, the cargo is a hydrophobic drug having a water solubility ranging from around 1 mg/mL to about 10 mg/mL. In some embodiments, the cargo is a hydrophobic drug having a water solubility ranging from around 0.1 mg/mL to about 1 mg/mL. In some embodiments, the cargo is a hydrophobic drug having a water solubility less than around 0.1 mg/mL. In some embodiments, the cargo is a prostaglandin-type therapeutic agent. Examples of prostaglandin-type therapeutic agents include, but are not limited to, latanoprost, bimatoprost, travaprost, tafluprost, unoprostone, and the like. Further prostaglandin-type therapeutic agents are described, for example, in U.S. Pat. Nos. 4,599,353; 5,321,128; 5,886,035; and 6,429,226, which patents are incorporated herein by reference in their entirety. Other suitable drugs for delivery using the compositions of the invention include, but are not limited to, carbonic anhydrase inhibitors, alpha agonists, beta blockers, cholinergic agents, antibiotics, antivirals, steroids, phosphodiesterase inhibitors (including, but not limited to, sildenafil), dilating agents, artificial tear agents for dry-eye, anti-allergy agents, antimetabolites, anti-inflammatory agents (including non-steroidal anti-inflammatory agents), and anti-VEGF agents.

The microparticles can further contain one or more additional UV blocking agents, analgesics (including opioid analgesics), anti-parasitics, anti-arrhythmic agents, anti-bacterial agents, anti-coagulants, anti-depressants, anti-diabetics, anti-epileptics, anti-fungal agents, anti-gout agents, anti-hypertensive agents, anti-malarials, anti-migraine agents, anti-muscarinic agents, anti-neoplastic agents, immunosuppressants, anti-protazoal agents, anti-thyroid agents anxiolytics, sedatives, hypnotics, neuroleptics, cardiac inotropic agents, corticosteroids, diuretics, anti-parkinsonian agents, gastro-intestinal agents, histamine H-receptor antagonists, lipid regulating agents, nitrates, anti-anginal agents, nutritional substances, sex hormones, and/or stimulants.

In some embodiments, the cargo is a drug for treating ocular diseases, such as latanoprost, dexamethasone, timolol (free base), timolol maleate, timolol hemihydrate, apraclonidine HCl, brimonidine (free base), brimonidine tartrate, betaxolol HCl, metipranolol, brinzolamide, methazolamide, dorzolamide, acetazolamide, pilocarpine HCl, carbachol, pilocarpine HCl, travaprost, bimatoprost, or tafluprost. In some embodiments the cargo is selected from bimatoprost, travaprost, tafluprost, acetazolamide, methazolamide, dorzolamide, brinzolamide, timolol, timolol acetate, pilocarpine, and combinations thereof. In some embodiments, the cargo is selected from latanoprost, dexamethasone, and combinations thereof. In some embodiments, the cargo is latanoprost.

In some embodiments, the cargo comprises a prostaglandin as described above, a carbonic anhydrase inhibitor, an alpha agonist, a beta blocker, a UV blocker, or a combination thereof. Examples of carbonic anhydrase inhibitors include, but are not limited to, acetazolamide, methazolamide, dorzolamide, and brinzolamide, as well as those disclosed in U.S. Pat. Nos. 5,153,192 and 4,797,413. Examples of alpha agonists include, but are not limited to, clonidine, apraclonidine, and brimonidine, as well as those described in U.S. Pat. Nos. 4,145,421 and 3,468,887. Examples of beta blockers include, but are not limited to, timolol, levobunolol, metipranolol, and carteolol, as well as those described in U.S. Pat. Nos. 4,061,636 and 3,655,663. Examples of UV blockers include, but are not limited to, avobenzone, octyl methoxycinnamate (octinoxate), octisalate, homosalate, octocrylene, para-aminobenzoic acid, cinoxate, oxybenzone (benzophenone-3), dioxybenzone (benzophenone-8), methyl anthranilate, octocrylene, padimate 0, ensulizole, sulisobenzone, trolamine salicylate, ecamsule, and the like.

The amount of the drug cargo in the microparticles will depend on factors such as the particular drug as well as the target dose and intended dosage regime. In general, the amount of cargo in the microparticles will range from about 0.1% (w/w) to about 50% (w/w), based on the total weight of the particles. The amount of cargo in the microparticles can range, for example, from about 0.1% (w/w) to about 1% (w/w), or from about 1% (w/w) to about 5% (w/w), or from about 5% (w/w) to about 10% (w/w), or from about 10% (w/w) to about 15% (w/w), or from about 15% (w/w) to about 20% (w/w), or from about 20% (w/w) to about 25% (w/w), or from about 25% (w/w) to about 30% (w/w), or from about 30% (w/w) to about 35% (w/w), or from about 35% (w/w) to about 40% (w/w), or from about 40% (w/w) to about 45% (w/w), or from about 45% (w/w) to about 50% (w/w). The amount of cargo in the microparticles can range from about 15% (w/w) to about 25% (w/w), or from about 10% (w/w) to about 30% (w/w), or from about 5% (w/w) to about 35% (w/w).

In some embodiments, the amount of cargo in the microparticles ranges from about 1% (w/w) to about 20% (w/w), based on the total weight of the microparticles. In some embodiments, the amount of cargo in the microparticles ranges from about 1% (w/w) to about 2% (w/w), or from about 2% (w/w) to about 3% (w/w), or from about 3% (w/w) to about 4% (w/w), or from about 4% (w/w) to about 5% (w/w), or from about 5% (w/w) to about 6% (w/w), or from about 6% (w/w) to about 7% (w/w), or from about 7% (w/w) to about 8% (w/w), or from about 8% (w/w) to about 9% (w/w), or from about 9% (w/w) to about 10% (w/w). In some embodiments, the amount of cargo in the microparticles ranges from about 10% (w/w) to about 11% (w/w), or from about 11% (w/w) to about 12% (w/w), or from about 12% (w/w) to about 13% (w/w), or from about 13% (w/w) to about 14% (w/w), or from about 14% (w/w) to about 15% (w/w), or from about 15% (w/w) to about 16% (w/w), or from about 16% (w/w) to about 17% (w/w), or from about 17% (w/w) to about 18% (w/w), or from about 18% (w/w) to about 19% (w/w), or from about 19% (w/w) to about 20% (w/w),

In some embodiments, the amount of cargo in the microparticles ranges from about 1% (w/w) to about 10% (w/w), or from about 2% (w/w) to about 9% (w/w), or from about 3% (w/w) to about 8% (w/w), or from about 4% (w/w) to about 7% (w/w). In some embodiments, the amount of cargo in the microparticles ranges from about 10% (w/w) to about 20% (w/w), or from about 12% (w/w) to about 18% (w/w), or from about 14% (w/w) to about 16% (w/w). In some embodiments, the amount of cargo in the microparticles ranges from about 2% (w/w) to about 18% (w/w), or from about 4% (w/w) to about 16% (w/w), or from about 6% (w/w) to about 14% (w/w), or from about 8% (w/w) to about 12% (w/w). In some embodiments, the amount of cargo in the microparticles is about 1, 1.5, 2, 2.5, 3, 3.5, 4, 4.5, 5, 5.5, 6, 6.5, 7, 7.5, 8, 8.5, 9, 9.5, 10, 10.5, 11, 11.5, 12, 12.5, 13, 13.5, 14, 14.5, 15, 15.5, 16, 16.5, 17, 17.5, 18, 18.5, 19, 19.5, or 20% (w/w).

Depending on the application, the cargo may include other particles. For example, particles having an average particle size ranging from 1 μm to 100 μm can act as suitable depots (i.e., composite particles) for containing other particles, preferably particles having smaller particle sizes, e.g., nanoparticles. These other smaller particles residing in the depot particles, can themselves contain a cargo, e.g., drugs for treating ocular diseases and/or other diseases.

In some embodiments, particles are coated with a mucoadhesive agent. Coating the particles of the present invention with mucoadhesive agents, which includes polymers, can increase the adhesion of particles to an ocular surface administered for the same time period. This can reduce the clearance of the formulation of the present invention from the eye. A number of suitable mucoadhesive agents can be used for coating the particles, including but not limited to poly(carboxylic acid-containing) based polymers, such as poly (acrylic, maleic, itaconic, citraconic, hydroxyethyl methacrylic or methacrylic) acid; gums such as xanthan gum, guar gum, locust bean gum, tragacanth gums, karaya gum, ghatti gum, cholla gum, psillium seed gum and gum arabic; clays such as montmorillonite clays and attapulgite clay; polysaccharides such as dextran, pectin, amylopectin, agar, mannan, polygalactonic acid, starches such as hydroxypropyl starch or carboxymethyl starch, and cellulose derivatives such as methyl cellulose, ethyl cellulose, hydroxypropylmethyl cellulose, and the like; polypeptides such as casein, gluten, gelatin, fibrin glue; chitosan, chitin, and salts or derivatives thereof such as chitosan lactate, chitosan glutamate, and carboxymethyl chitin; glycosaminoglycans such as hyaluronic acid (also called hyaluronan); metals or water soluble salts of alginic acid such as sodium alginate or magnesium alginate. In some embodiments, the mucoadhesive agent is chitosan, also known as deacetylated chitin or poly(D-glucosamine). In some embodiments, the molecular weight of the chitosan ranges from about 40 kDa to about 400 kDa. The molecular weight of the chitosan can range, for example, from about 40 kDa to about 200 kDa, or from about 50 kDa to about 190 kDa, or from about 200 kDa to about 400 kDa, or from about 300 kDa to about 400 kDa, or from about 310 kDa to about 375 kDa. The molecular weight of chitosan can be determined by measuring the viscosity of a chitosan solution (e.g., 1% (w/w) chitosan in 1% acetic acid at 25° C.) as previously described (e.g., by Roberts. International Journal of Biological Macromolecules. 1982: 4, 374-377.

In some embodiments, the mucoadhesive coating constitutes from about 0.01% (w/w) to about 5% (w/w) of the total mass of the microparticles. In some embodiments, the mucoadhesive coating constitutes 1% (w/w) or less of the total mass of the microparticles. For example, the amount of the mucoadhesive coating (e.g., chitosan) can range from about 0.01% (w/w) to about 0.05% (w/w), or from about 0.05 (w/w) to about 0.1% (w/w), or from about 0.1% (w/w) to about 0.25% (w/w), or from about 0.25% (w/w) to about 0.5% (w/w), or from about 0.5% (w/w) to about 0.75% (w/w), or from about 0.75% (w/w) to about 1% (w/w). The amount of the mucoadhesive coating (e.g., chitosan) can range from about 0.05% (w/w) to about 0.95% (w/w), or from about 0.1 (w/w) to about 0.9% (w/w), or from about 0.2% (w/w) to about 0.8% (w/w), or from about 0.4% (w/w) to about 0.6% (w/w).

In some embodiments, the invention provides a composition comprising a population of particles having an average particle size, wherein the particles are adapted to carry and release a cargo upon topical ocular administration to a subject, wherein the cargo comprises latanoprost and/or dexamethasone, and wherein the average particle size of at least one of the populations of particles ranges from about 1 μm to about 25 μm. In some such embodiments, the particles comprise poly(lactic-co-glycolic acid) having a molecular weight ranging from about 25 kDa to about 125 kDa. In some such embodiments, the molar ratio of lactic acid to glycolic acid in the poly(lactic-co-glycolic acid) ranges from about 40:60 to about 60:40. In some embodiments, the molar ratio of lactic acid to glycolic acid in the poly(lactic-co-glycolic acid) is about 50:50. In some embodiments, the molecular weight of the poly(lactic-co-glycolic acid) ranges from about 30 kDa to about 60 kDa and the molar ratio of lactic acid to glycolic acid in the poly(lactic-co-glycolic acid) is about 50:50. In some embodiments, the molecular weight of the poly(lactic-co-glycolic acid) ranges from about 7 kDa to about 17 kDa and the molar ratio of lactic acid to glycolic acid in the poly(lactic-co-glycolic acid) is about 50:50. In some embodiments, the molecular weight of the poly(lactic-co-glycolic acid) ranges from about 66 kDa to about 107 kDa and the molar ratio of lactic acid to glycolic acid in the poly(lactic-co-glycolic acid) is about 75:25.

### III. Preparation of Microparticles

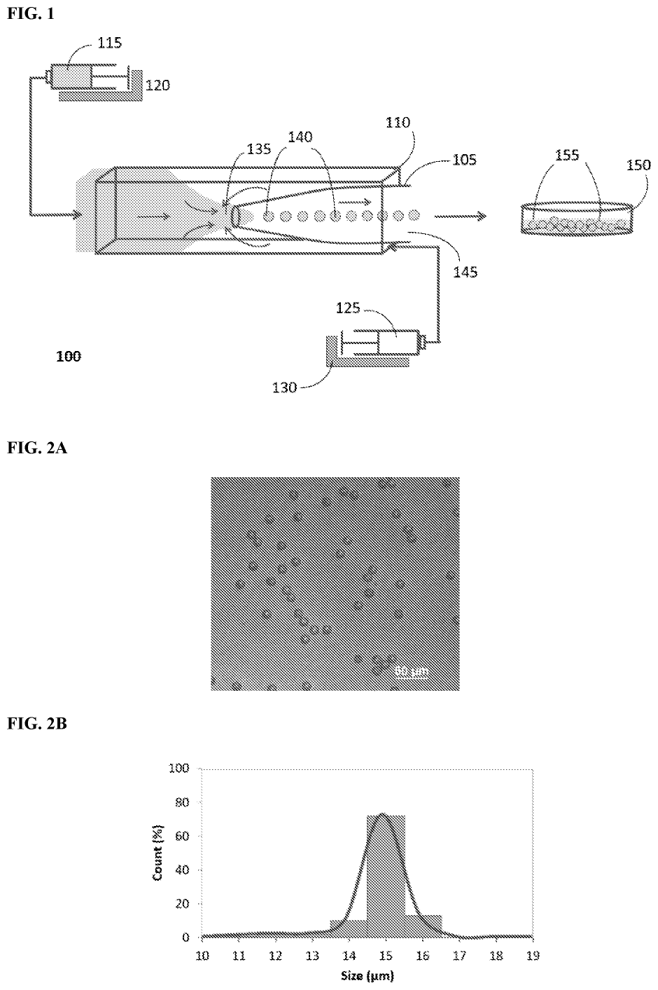

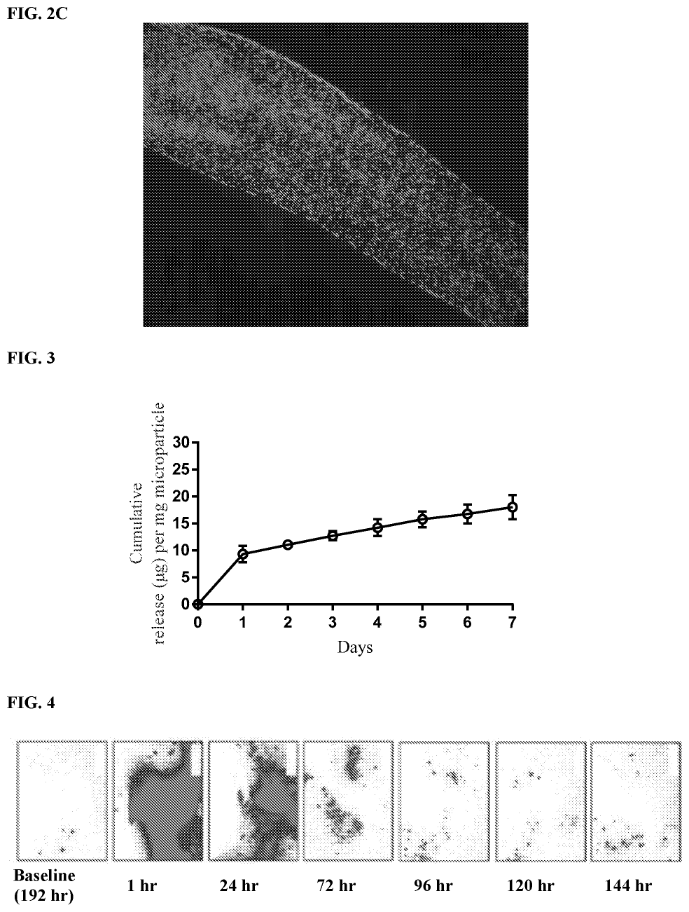

Microfluidics techniques, e.g., capillary microfluidic techniques, can be used to manufacture the particles of the present invention. Capillary microfluidic techniques have shown to be capable of scalable manufacture of highly monodisperse polymeric particles (FIG. 2A) with precise control over the size of final particles and drug loading. An example of a capillary microfluidic technique for the manufacture of the particles of the present invention is shown in FIG. 1, where fluids in the dispersed phase are hydrodynamically flow focused through the nozzle of a capillary to form emulsion droplets which are collected, and where solvent evaporation occurs after collection to yield the desired particles. An example of a capillary microfluidic technique will be discussed in further detail below.

As shown in FIG. 1, a coaxial capillary assembly 100 is assembled by positioning a round capillary 105 inside a square capillary 110. An organic phase 115 is introduced into one end of the square capillary via syringe pump 120, or other suitable means (e.g., a peristaltic pump), at a first flow rate while an aqueous phase 125 is introduced into the opposite end of the square capillary by syringe pump 130, or other suitable means, at a second flow rate. The aqueous phase and the organic phase are introduced into the void space between the exterior of the circular capillary and the interior of the square capillary. The phases meet at aperture 135 on one end of the circular capillary, causing the formation of emulsion droplets 140, which travel through the circular capillary and exit at opening 145 for collection in plate 150 or another suitable receptacle. Evaporation of liquids from the material yields microparticles 155.

The capillaries can be fashioned from any suitable material, particularly those which are normally associated with microfabrication techniques including, e.g., silica based substrates, such as glass, quartz, silicon or polysilicon, as well as other substrate materials, such as gallium arsenide and the like. One or more coating layers, e.g., silicon oxide, can be applied over the interior and/or exterior surfaces. Capillaries can also be coated with plastics such as polymethylmethacrylate (PMMA), polycarbonate, polytetrafluoroethylene (TEFLON™), polyvinylchloride (PVC), polydimethylsiloxane (PDMS), poly ether ether ketone (PEEK), polysulfone, and the like. Capillary surfaces can also be hydrophilized to increase hydrophilicity, e.g., by treatment with oxygen plasma or nitrogen plasma using known techniques and devices such as a BT-1 plasma processing system (Plasma Etch, Inc.) or a PC-1100 plasma cleaning system (SAMCO Inc.).

The organic phase used for preparing the microspheres contains a biocompatible matrix material (e.g., a polymer), a cargo material (e.g., an ophthalmic therapeutic agent), an optional components (e.g., a pharmaceutical excipient) dissolved or otherwise disperse in an organic solvent. Any suitable organic solvent can be used for forming the organic phase, provided that it is immiscible with the aqueous phase. Examples of suitable organic solvents include, but are not limited to, ethyl acetate, toluene, benzene, chloroform, carbon tetrachloride, dichloromethane, 1,2-dichloroethane, diethyl ether, methyl-tert-butyl ether, heptane, hexane, pentane, cyclohexane, petroleum ether, and combinations thereof.

The organic phase can contain any suitable amount of matrix material and cargo. The organic phase will typically contain a polymer or other matrix material in amounts ranging from about 0.01% (w/w) to about 10% (w/w). The concentration of polymer in the organic phase can range, for example, from about 0.01% (w/w) to about 0.05% (w/w), or from about 0.05% (w/w) to about 0.1% (w/w), or from about 0.1% (w/w) to about 0.25% (w/w), or from about 0.25% (w/w) to about 0.5% (w/w), or from about 0.5% (w/w) to about 1% (w/w), or from about 1% (w/w) to about 2.5% (w/w), or from about 2.5% (w/w) to about 5% (w/w), or from about 5% (w/w) to about 10% (w/w). The concentration of polymer in the organic phase can range from about 0.01% (w/w) to about 9.9% (w/w), or from about 0.05% (w/w) to about 7.5% (w/w), or from about 0.1% (w/w) to about 5% (w/w), or from about 0.25% (w/w) to about 2.5% (w/w). The amount of the polymer in the organic phase can range from about 0.6% (w/w) to about 0.8% (w/w), or from about 0.4% (w/w) to about 0.8% (w/w), or from about 0.2% (w/w) to about 1% (w/w), or from about 0.1% (w/w) to about 1.5% (w/w), or from about 0.05% to about 2.5% (w/w), or from about 0.01% (w/w) to about 3% (w/w). One of skill in the art will appreciate that the concentration of the polymer in the organic phase can be expressed in different units and will be able to ready convert between units. In the case of an organic solution containing a polymer and dichloromethane, for example, one of skill in the art will understand that a polymer concentration of about 0.01-3% (w/w) amounts to a concentration of about 0.113-39.9 mg/mL. One of skill in the art will further be able to factor in the amounts of active agents and optional components (e.g., excipients) in order to convert between units of concentration. The total amount of polymer or other matrix material in the organic phase will depend, in part, on factors such as the identity of the polymer and the solvent as well as the particular cargo and content of the aqueous phase.

The organic phase will typically contain an ophthalmic therapeutic agent or other cargo material in amounts ranging from about 0.01% (w/w) to about 10% (w/w). The concentration of ophthalmic therapeutic agent in the organic phase can range, for example, from about 0.01% (w/w) to about 0.05% (w/w), or from about 0.05% (w/w) to about 0.1% (w/w), or from about 0.1% (w/w) to about 0.25% (w/w), or from about 0.25% (w/w) to about 0.5% (w/w), or from about 0.5% (w/w) to about 1% (w/w), or from about 1% (w/w) to about 2.5% (w/w), or from about 2.5% (w/w) to about 5% (w/w), or from about 5% (w/w) to about 10% (w/w). The concentration of ophthalmic therapeutic agent in the organic phase can range from about 0.01% (w/w) to about 9.9% (w/w), or from about 0.05% (w/w) to about 7.5% (w/w), or from about 0.1% (w/w) to about 5% (w/w), or from about 0.25% (w/w) to about 2.5% (w/w). The amount of ophthalmic therapeutic agent in the organic phase can range from about 0.01% (w/w) to about 0.02% (w/w), or from about 0.02% (w/w) to about 0.04% (w/w), or from about 0.04% (w/w) to about 0.06% (w/w), or from about 0.06% (w/w) to about 0.08% (w/w), or from about 0.08% to about 0.1% (w/w), or from about 0.1% (w/w) to about 0.12% (w/w), or from about 0.12% (w/w) to about 0.14% (w/w), or from about 0.14% (w/w) to about 0.16% (w/w), or from about 0.16% (w/w) to about 0.18% (w/w). One of skill in the art will be able to make conversions between concentration units as described above. The total amount of ophthalmic therapeutic agent or other cargo in the organic phase will depend, in part, on factors such as the identity of the cargo and the solvent as well as the particular matrix material and content of the aqueous phase.

In certain embodiments, the organic phase contains a biodegradable polymer and one or more prostaglandin-type therapeutic agents dissolved in an organic solvent. In some such embodiments, the biodegradable polymer is PLGA as described above. In some such embodiments, the ratio of lactic acid to glycolic acid in the PLGA is 50:50. In some such embodiments, the molecular weight of the PLGA ranges from about 25 g/mol to about 125,000 g/mol. In some embodiments, the organic phase contains PLGA (e.g., 50:50 PLGA, 30,000-60,000 g/mol; or 50:50 PLGA, 7,000-17,000 g/mol); and one or more ophthalmic agents selected from bimatoprost, latanoprost, tafluprost, and travoprost; and an organic solvent. In some embodiments, the organic phase contains PLGA (e.g., 50:50 PLGA, 30,000-60,000 g/mol; or 50:50 PLGA, 7,000-17,000 g/mol); and one or more prostaglandin-type therapeutic agents (e.g., latanoprost); and an organic solvent selected from chloroform, carbon tetrachloride, dichloromethane, and 1,2-dichloroethane. In some such embodiments, the organic solvent is dichloromethane.

In some embodiments, the organic phase contains PLGA (e.g., 50:50 PLGA, 30,000-60,000 g/mol; or 50:50 PLGA, 7,000-17,000 g/mol) in an amount ranging from 0.01% (w/w) to about 3% (w/w), latanoprost in an amount ranging from about 0.01 (w/w) to about 0.18% (w/w), and dichloromethane. In some embodiments, the organic phase contains PLGA in amount ranging from about 0.6% (w/w) to about 0.9% (w/w) and latanoprost in an amount ranging from about 0.14% (w/w) to about 0.16% (w/w). In some embodiments, the organic phase contains about 0.75% (w/w) PLGA (e.g., 50:50 PLGA, 30,000-60,000 g/mol; or 50:50 PLGA, 7,000-17,000 g/mol); about 0.15% (w/w) latanoprost; and about 99.1% (w/w) dichloromethane.

The aqueous phase used for preparing the microspheres contains water, and the aqueous phase can optionally contain additional components. The aqueous phase can contain, for example, one or more buffers, cosolvents, salts, detergents/surfactants, and/or chelators. Examples of suitable buffers include, but are not limited to 2-(N-morpholino)ethanesulfonic acid (MES), 2-[4-(2-hydroxyethyl)piperazin-1-yl]ethanesulfonic acid (HEPES), 3-morpholinopropane-1-sulfonic acid (MOPS), 2-amino-2-hydroxymethyl-propane-1,3-diol (TRIS), potassium phosphate, sodium phosphate, phosphate-buffered saline, sodium citrate, sodium acetate, sodium borate, and the like. Examples of suitable cosolvents include, but are not limited to, dimethylsulfoxide, dimethylformamide, ethanol, methanol, tetrahydrofuran, acetone, acetic acid, and the like. Examples of suitable osmogents include, but are not limited to, carbohydrates (e.g., xylitol, mannitol, sorbitol, sucrose, dextrose, and the like); urea and derivatives thereof; and water-soluble polymers (e.g., poly(ethylene glycol), hydroxypropylmethyl cellulose, poly(vinylalcohol), poly(acrylic acid), poly(methylacrylic acid), poly(styrenesulfonic acid), and the like). Examples of suitable detergents/surfactants include, but are not limited to, non-ionic surfactants such as N,N-bis[3-(D-gluconamido)propyl]cholamide, polyoxyethylene (20) cetyl ether, dimethyldecylphosphine oxide, branched octylphenoxy poly(ethyleneoxy)ethanol, a polyoxyethylene-polyoxypropylene block copolymer, t-octylphenoxypolyethoxyethanol, polyoxyethylene (20) sorbitan monooleate, and the like; anionic surfactants such as sodium cholate, N-lauroylsarcosine, sodium dodecyl sulfate, and the like; cationic surfactants such as hexdecyltrimethyl ammonium bromide, trimethyl(tetradecyl) ammonium bromide, and the like; and zwitterionic surfactants such as an amidosulfobetaine, 3-[(3-cholamidopropyl)dimethyl-ammonio]-1-propanesulfonate, and the like). Examples of suitable chelators include, but are not limited to, ethylene glycol-bis(2-aminoethylether)-N,N,N′,N′-tetraacetic acid (EGTA), 2-({2-[bis(carboxymethyl)amino]ethyl}(carboxymethyl)amino)acetic acid (EDTA), 1,2-bis(o-aminophenoxy)ethane-N,N,N′,N′-tetraacetic acid (BAPTA)), and the like.

Buffers, cosolvents, osmogents, salts, detergents/surfactants, and chelators can be used at any suitable concentration, which can be readily determined by one of skill in the art. In general, buffers, cosolvents, osmogents, salts, detergents/surfactants, and chelators are included in reaction mixtures at concentrations ranging from about 0.001% (w/w) to about 10% (w/w), e.g., from about 0.01% (w/w) to about 1% (w/w). For example, a buffer, a cosolvent, an osmogent, a salt, a detergent/surfactant, or a chelator can be included in the aqueous phase at a concentration of about 0.001% (w/w), or about 0.01% (w/w), or about 0.1% (w/w), or about 1% (w/w), or about 10% (w/w). In some embodiments, the aqueous phase comprises water and a water-soluble polymer. In some embodiments, the aqueous phase comprises a water-soluble polymer in an amount ranging from about 0.5% (w/w) to about 5% (w/w). In some embodiments, the aqueous phase comprises poly(vinyl alcohol) in an amount ranging from about 0.5% (w/w) to about 5% (w/w). In some embodiments, the aqueous phase comprises poly(vinyl alcohol) in an amount of around 1% (w/w).

Returning to FIG. 1, the flow rate of the organic phase and aqueous phase through the void space between the exterior of the circular capillary and the interior of the square capillary can be controlled to focus the flow of the fluid through the aperture of the circular capillary 135 and to vary the size of the emulsion droplets 140 that are formed. The difference between the flow rate of the aqueous phase and the flow rate of the organic phase can depend, in part, on factors such as the dimensions of the capillaries and the particular components in the aqueous phase and the organic phase. In some embodiments, the flow rate of the aqueous phase through the coaxial capillary assembly will be greater than the flow rate of the organic phase through the coaxial capillary assembly. In some embodiments, the flow rate of the aqueous phase will be less than the flow rate of the organic phase. In some embodiments, the flow rate of the aqueous phase and the flow rate of the organic phase will be equal. The flow rate of either phase will typically range from a few microliter per minute (μL/min) to tens of milliliters per minute (mL/min) depending on factors such and dimensions of the capillaries and the particular components in the aqueous phase and the organic phase. In some embodiments, the aqueous phase is introduced into the capillary system at a flow rate ranging from about 50 μL/min to about 500 μL/min (e.g., from about 100 μL/min to about 125 μL/min, or from about 75 μL/min to about 250 μL/min). In some such embodiments, the organic phase is introduced into the capillary system at a flow rate ranging from about 1 μL/min to about 100 μL/min (e.g., from about 15 μL/min to about 30 μL/min, or from about 5 μL/min to about 50 μL/min). In some embodiments, an aqueous phase comprising water and poly(vinyl alcohol) is introduced into the capillary system at a flow rate ranging from about 110 μL/min to about 120 μL/min, and organic phase comprising dichloromethane and latanoprost introduced into the capillary system at a flow rate ranging from about 10 μL/min to about 25 μL/min).

The dimensions of the emulsion droplets formed upon contact of the aqueous phase and the organic phase will depend on factors such as flow rates of the two phases and the dimensions of the external capillary and the internal capillary. Typically, the diameter of the emulsion droplets will range from about 5 μm to about 500 μm (e.g., from about 10 μm to about 250 μm). A liquid phase (e.g., an aqueous solution) can be added to plate 150 in order to prevent aggregation of emulsion droplets after they are collected from the capillary assembly. The liquid phase can further contain a portion of dissolved cargo material, in order to reduce or eliminate diffusion of the cargo from the emulsion droplets during evaporation to form the final microparticles. Evaporation can be conducted at room temperature (i.e., 20-25° C.) or at elevated temperatures (e.g., 40-60° C.) for a period of time sufficient to remove enough of the organic phase and aqueous phase for the microparticles to solidify. Typically, evaporation will be conducted for periods of time ranging from a few minutes to several hours depending on factors, such as the volume of material to be evaporated, the temperature during evaporation, and the relative humidity during evaporation. Following evaporation, the resulting microparticles can be optionally washed with one or more portions of water, or another suitable solvent, to remove residual amounts of the aqueous and/or organic phases. The microparticles can then be collected (e.g., by centrifugation, filtration, or other means), and optionally dried prior to use.

The microparticles can be coated with a mucoadhesive agent as described above. The coating can be applied by suspending the microparticles in a solution of the mucoadhesive agent. For example, the mucoadhesive agent (e.g., chitosan, hyaluronan, or another polymer) can be dissolved at a concentration of 0.01-10% (w/w) (e.g., 1% w/w) and combined in suspension with the microparticles at room temperature for a period of time ranging from a few minutes to several hours. The particles can then be collected, optionally washed, and optionally dried as described above.

### IV. Solid Polymer Matrix Formulations

In a related aspect, the invention provides a composition comprising a population particles as described above and a solid polymer matrix. The particles of the invention are partially or fully embedded in a solid polymer matrix, which can be administered via direct topical application to a target tissue or organ (e.g., the conjunctiva of an eye). Typically, the solid polymer matrix will be hydrophilic in nature, providing for gelation, partial dissolution, and/or full dissolution upon contact with bodily fluids and subsequent delivery of the particles to the target. Examples of suitable materials for inclusion in the solid polymer matrix include, but are not limited to, poly(ethylene glycol), polyvinyl pyrrolidone, polyvinyl alcohol, polyacrylic acid, polyacrylamide, poly(N-2-hydroxypropyl) methylacrylamide), poly(methyl vinyl ether-alt-maleic anhydride), and poly(2-alkyl-2-oxazolines) such as poly(2-ethyl-2-oxazoline).

In certain embodiments, the solid polymer formulations are provided as flakes (or laminae) consisting of a thin layer of particles embedded fully or partially in the polymer matrix. Typically, the thickness of the flake will range from about 1 μm to about 500 μm. Thicknesses around 100 μm or less can be particularly advantageous for administration to the eyes of a subject without causing foreign body sensations or undue discomfort in the subject. The length and width of the flakes can vary depending on factors such as the intended target tissue/organ and the composition of the solid polymer matrix. In certain embodiments, the length and/or the width of the flake is less than 20 mm, e.g., less than 15 mm, less than 12 mm, or less than 10 mm. In some embodiments, the length and/or width of the flake ranges from about 1 mm to about 10 mm. In some embodiments, the thickness of the flake ranges from about 1 μm to about 100 μm, the length of the flake ranges from about 1 mm to about 10 mm, and the width of the flake ranges from about 1 mm to about 10 mm.

In general, the amount of microparticles in the solid polymer formulation will range from about 5% (w/w) to about 99% (w/w), based on the total weight of the formulation. The amount of cargo in the microparticles can range, for example, from about 5% (w/w) to about 10% (w/w), or from about 10% (w/w) to about 20% (w/w), or from about 20% (w/w) to about 30% (w/w), or from about 30% (w/w) to about 40% (w/w), or from about 40% (w/w) to about 50% (w/w), or from about 50% (w/w) to about 60% (w/w), or from about 60% (w/w) to about 70% (w/w), or from about 70% (w/w) to about 80% (w/w), or from about 80% (w/w) to about 90% (w/w), or from about 90% (w/w) to about 99% (w/w). The amount of cargo in the microparticles can range from about 50% (w/w) to about 99% (w/w), or from about 60% (w/w) to about 99% (w/w), or from about 70% (w/w) to about 99% (w/w), or from about 80% (w/w) to about 99% (w/w).

Advantageously, the solid polymer formulations provide prolonged ocular residence times of microparticle cargo (e.g., therapeutic agents, UV blockers, etc.). The formulations can provide delivery of cargo for periods of time ranging from hours to days, e.g., 1 hour, 2 hours, 4 hours, 6 hours, 12 hours, 18 hours, 24 hours, 2 days, 3 days, 4 days, 5 days, 6 days, 7 days, 8 days, 9 days, 10 days, 14 days, 21 days, 28 days, or longer. The ocular residence time of a therapeutic agent or other cargo will depend, in part, on factors such as the size of the flakes in the composition as well as the content of the solid polymer matrix. As a non-limiting example, the molecular weight of a polymer (e.g., polyvinyl alcohol) in the solid polymer matrix can be varied to control the rate at which the matrix disintegrates or otherwise disperses following application to a target tissue such as the conjunctiva.

The solid polymer formulations can optionally comprise one or more additional excipients. When present, excipients are typically included in amounts that do not substantially affect the solidification of the polymer matrix or the rate at which the matrix disintegrates or otherwise disperses following topical administration. In certain embodiments, for example, the combined mass of excipients will not exceed 10% (w/w) of the solid polymer formulation. In some embodiments, the combined mass of excipients amounts to no more than 5% (w/w) of the solid formulation (e.g., 1% (w/w) or less). Suitable excipients include, but are not limited to, demulcents for reduction of irritation, tonicity agents, preservatives, chelating agents, buffering agents, surfactants, solubilizing agents, stabilizing agents, comfort-enhancing agents, polymers, emollients, pH-adjusting agents, and/or lubricants. Suitable demulcents include, but are not limited to, glycerin, polyvinyl pyrrolidone, poly(ethylene glycol) (e.g., poly(ethylene glycol) 400, propylene glycol, and polyacrylic acid. Suitable tonicity-adjusting agents include, but are not limited to, mannitol, sodium chloride, glycerin, and the like. Suitable buffering agents include, but are not limited to, phosphates, acetates and the like, and amino alcohols such as 2-amino-2-methyl-1-propanol (AMP). Suitable surfactants include, but are not limited to, ionic and nonionic surfactants (though nonionic surfactants are preferred), RLM 100, POE 20 cetylstearyl ethers such as Procol® C S20, poloxamers such as Pluronic® F68, and block copolymers such as poly(oxyethylene)-poly(oxybutylene). Suitable preservatives include, but are not limited to, p-hydroxybenzoic acid ester, sodium perborate, sodium chlorite, alcohols such as chlorobutanol, benzyl alcohol or phenyl ethanol, guanidine derivatives such as polyhexamethylene biguanide, sodium perborate, polyquaternium-1, or sorbic acid.

### V. Methods of Administration and Treatment

The composition of the present invention can be in the form of a solution or a gel. The composition may further comprise suitable excipients. The composition of the present invention can be applied topically to the eye in the form of an eye drop, ointment, and/or lotion.

Microparticles of the invention can be formulated as suspensions in a suitable fluid medium. The fluid medium can optionally comprise one or more additional excipients. Suitable excipients include, but are not limited to, demulcents for reduction of irritation, tonicity agents, preservatives, chelating agents, buffering agents, surfactants, solubilizing agents, stabilizing agents, comfort-enhancing agents, polymers, emollients, pH-adjusting agents, and/or lubricants. Suitable demulcents include, but are not limited to, glycerin, polyvinyl pyrrolidone, poly(ethylene glycol) (e.g., poly(ethylene glycol) 400, propylene glycol, and polyacrylic acid. Suitable tonicity-adjusting agents include, but are not limited to, mannitol, sodium chloride, glycerin, and the like. Suitable buffering agents include, but are not limited to, phosphates, acetates and the like, and amino alcohols such as 2-amino-2-methyl-1-propanol (AMP). Suitable surfactants include, but are not limited to, ionic and nonionic surfactants (though nonionic surfactants are preferred), RLM 100, POE 20 cetylstearyl ethers such as Procol® CS20, poloxamers such as Pluronic® F68, and block copolymers such as poly(oxyethylene)-poly(oxybutylene). Suitable preservatives include, but are not limited to, p-hydroxybenzoic acid ester, sodium perborate, sodium chlorite, alcohols such as chlorobutanol, benzyl alcohol or phenyl ethanol, guanidine derivatives such as polyhexamethylene biguanide, sodium perborate, polyquaternium-1, or sorbic acid.

Formulations of the present invention are preferably isotonic, or slightly hypotonic in order to prevent tears and eye tissue from becoming hypertonic. Accordingly, it can be advantageous for the osmolality of the formulation to be in the range of 210 to 320 milliosmoles per kilogram (mOsm/kg) (e.g., 220-320 mOsm/kg, or 235-300 mOsm/kg). The ophthalmic formulations can be formulated as sterile aqueous solutions.

Formulations of the invention can be provided as a ready-made suspension containing the microparticles dispersed in a fluid medium as described above. Alternatively, microparticles can be provide in a kit containing a fluid medium for suspension of the particles in a final formulation. The fluid medium can contain any of the demulcents, tonicity agents, preservatives, chelating agents, buffering agents, surfactants, solubilizing agents, stabilizing agents, comfort-enhancing agents, polymers, emollients, pH-adjusting agents, and/or lubricants described above. Kits according to the invention can contain the microparticles and the fluid medium in any of a number of suitable packaging forms including, but not limited to, vials, ampules, syringes, and capsules.

The particles and compositions of the invention can be administered to treat ocular diseases and/or disorders, in particular intraocular diseases and/or disorders. Such ocular diseases and/or disorders include but are not limited to glaucoma (includes primary angle-closure glaucoma), conjunctivitis and dry eyes.

In certain embodiments, the invention provides a method for treating glaucoma. The method includes topically administering an effective amount of a composition as described herein to the eyes of a subject having glaucoma. The amount of the composition can vary, depending in part on factors such as the severity of the glaucoma and the particular drug cargo contained in the microparticles. The composition can be administered such that from about 0.1 to about 500 μg (e.g., 0.1-200 μg) of the drug is administered to eye tissue per day. The amount of drug delivered to the eye can range, for example, from about 0.1 μg/day to about 1 μg/day, or from about 1 μg/day to about 10 μg/day, or from about 10 μg/day to about 20 μg/day, or from about 20 μg/day to about 30 μg/day, or from about 30 μg/day to about 40 μg/day, or from about 40 μg/day to about 50 μg/day, or from about 50 μg/day to about 60 μg/day, or from about 60 μg/day to about 70 μg/day, or from about 70 μg/day to about 80 μg/day, or from about 80 μg/day to about 90 μg/day, or from about 90 μg/day to about 100 μg/day. The amount of drug delivered to the eye can range from about 100 μg/day to about 150 μg/day, or from about 150 μg/day to about 200 μg/day, or from about 200 μg/day to about 250 μg/day, or from about 250 μg/day to about 300 μg/day, or from about 300 μg/day to about 350 μg/day, or from about 350 μg/day to about 400 μg/day, or from about 400 μg/day to about 450 μg/day, or from about 450 μg/day to about 500 μg/day.

In some embodiments, the amount of drug delivered to the eye ranges from about 5 μg/day to about 15 μg/day, or from about 1 μg/day to about 20 μg/day, or from about 1 μg/day to about 50 μg/day, or from about 1 μg/day to about 100 μg/day, or from about 1 μg/day to about 150 μg/day. In some embodiments, the amount of drug delivered to the eye is about 1, 2, 3, 4, 5, 6, 7, 8, 9, 10, 11, 12, 13, 14, 15, 16, 17, 18, 19, 20, 21, 22, 23, 24, 25, 30, 35, 40, 45, 50, 55, 60, 65, 70, 75, 80, 85, 90, 95, 100, 150, 200, 250, 300, 350, 400, 450, or 500 μg/day. Microparticles according to the invention can be administered in the form of a suspension as described above, usually in volumes ranging from a few microliters (μL) to a few hundred μL.

Advantageously, the compositions of the invention provide extended ocular persistence of the drug such that drug doses ranging from 0.1 μg/day to about 100 μg/day, or higher, can be provided over an extended period of following a single administration of the composition. For example, administration of a single 30-4, dose of a microparticle suspension according to the invention can provide delivery of the drug in an amount of 0.1-100 μg/day (e.g., 2-20 μg/day) for 1 hour, 2 hours, 4 hours, 6 hours, 8 hours, 12 hours, 18 hours, 24 hours, 36 hours, 2 days, 3 days, 4 days, 5 days, 6 days, 7 days, 8 days, 9 days, 10 days, 14 days, 21 days, 28 days, or longer. In some embodiments, the methods of the invention include topically administering a suspension of microparticles containing PLGA and latanoprost to the eyes of a subject in need thereof, wherein the suspension is administered as a single dose in an amount sufficient to deliver latanoprost to the eyes in an amount ranging from about 2 μg/day to about 20 μg/day over a period of at least seven days.

### VI. Examples

### Example 1. Preparation of Latanoprost-Loaded Microparticles

Organic/water (O/W) emulsions were generated using a glass capillary microfluidic setup as shown in FIG. 1. An axisymmetric coaxial glass capillary focusing device was assembled using a square and round capillary. The surface of round capillary was hydrophilized by treatment with oxygen plasma (100 W) for 120 s. The aqueous continuous phase (W) was prepared by mixing PVA (67,000 g/mol; 1% w/v) in distilled water. The disperse phase (0) was prepared by dissolving 50 mg of 50:50 PLGA (30,000-60,000 g/mol) and 10 mg of the hydrophobic drug, latanoprost, in 5 mL dichloromethane for 15 min, followed by filtration with a 0.22 μm PTFE syringe filter. W and O phases were infused from the two ends of the square capillary through the outer coaxial region using two syringe pumps at flow rates of 115 μL/min and 20 μL/min, respectively. The fluids were hydrodynamically flow focused through the nozzle of the round capillary resulting in the formation of the emulsion droplets. 6 cm ID glass wells were used for sample collection. Approximately 125 μL of O/W emulsions were dispersed directly into the glass well containing a pre-dispensed film (1.0 mm) of latanoprost solution (30 μg/mL). Solvent evaporation was performed at room temperature (25° C.) for 1 hr. The particles were washed three times using distilled water, placed in a pre-weighed vial and dried in vacuum for 3 hr. The weight of the particles was measured after desiccation.

### Example 2. Preparation of Chitosan-Coated Latanoprost-Loaded Microparticles

Dried particles were prepared according to Example 1 and resuspended in 0.5% low molecular weight chitosan (50-190 kDa, as determined by viscosity measurements) in phosphate buffered saline, pH 5.5 (adjusted with glacial acetic acid) or water, pH 5 (adjusted with 1M sodium hydroxide). The suspension was incubated overnight at 4° C. The final microparticle suspension ranged in concentration from 8% to 10% (v/v) and in size from 11 μm to 16 μm (average 15 μm), with a Zeta potential of +20 mV. Suspensions ranging in concentration from 20-25% were also prepared.

### Example 3. Drug Release from Microparticles

Known weights of particle samples were added to 1 mL of PBS buffer in a vial and shaken at 225 rpm at 37° C. 1×PBS (phosphate buffered saline) solution (pH 7.4) was used as the release medium. Varied shaking rates (e.g., 150 rpm) and temperature (e.g., room temperature) were used in certain instances. Two 100 μL samples were withdrawn at intervals of 24 hours for up to 7 days and fresh PBS buffer was added to the solution in order to keep the volume of the release medium constant. Release samples were prepared by mixing equal volumes of 50% release medium and 50% acetonitrile. The use of acetonitrile as the mobile carrier does not influence the degradation of PLGA microparticles since the release sample is a supernatant of microparticles in PBS buffer.

HPLC analysis was carried out on HP series 1100 HPLC-VWD analyzer. The separation was performed on a C-18 chromatography column (Agilent C18, 2.7 μM, 4.6×150 mm) at 25° C. using water and acetonitrile in the ratio of 30:70 (v/v) as the mobile phase. The flow rate of the mobile phase was set at 1 mL/min and the detector wavelength was set at 210 nm. A characteristic retention time of 3 min was observed for latanoprost under these conditions. As shown in FIG. 3, a slow release trend was observed with an initial steep increase within the first 2-3 days.

### Example 4. Ocular Residence Time and Drug Release in Rabbit Eyes

Chitosan-coated PLGA particles loaded with red fluorescent dye (Nile red) were prepared according to Example 2 and Example 3, and the particles were administered to rabbits (4 animals, 8 eyes). A one week study was conducted with four rabbits. An ocular residence time of up to 7 days was achieved. Images of rabbit eye taken before and after administration of the dye-loaded formulation are shown in FIG. 4. Microparticles were retained in the preocular area for up to one week, without any signs of ocular inflammation or other adverse effects.

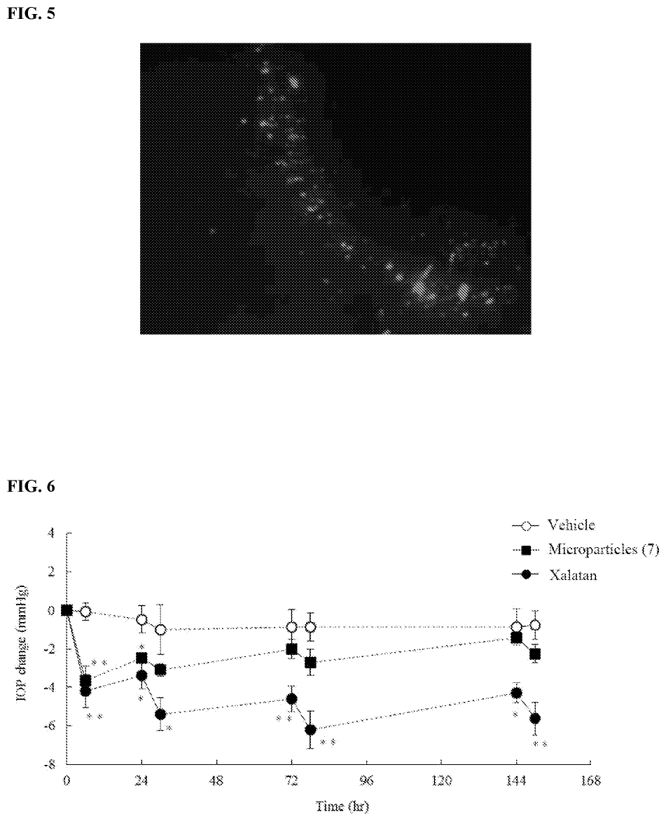

Chitosan-coated PLGA particles loaded with Nile red (0.1 mg/mL) and latanoprost (2 mg/mL) were prepared according to Example 2 and Example 3, and the particles were administered to rabbits (4 animals, eight eyes). A statistically significant level of released latanoprost was observed in rabbit tear fluid over a period of one week, as summarized in Table 1. Microparticles were retained in the preocular area for up to 7 days, as shown in FIG. 5.

TABLE 1

| | Baseline | Day 5 | Day 7 |
|---|---|---|---|
| Latanoprost in | 0 | 781 | 1132 |
| tear fluid (ng/mL) | | | (P value < 0.05) |

The safety of the formulations was studied over a 5-week period in rabbits (4 animals, 8 eyes). The formulations were administered once every 7 days. No sign of local ocular inflammation or other adverse effects were observed over the 5-week period, and intraocular pressure remained normal. Lowering of intraocular pressure under the experimental conditions was not expected because the rabbits used in the study do not respond to latanoprost. Taken together, the results of the study indicate that the test formulation provides sustained release of latanoprost without adverse effects.

### Example 5. Preparation of Nile Red-Loaded Microspheres

O/W emulsions were generated using a glass capillary microfluidic setup as shown in FIG. 1. The axisymmetric coaxial glass capillary now-focusing device was assembled using a square and round capillary. The surface of round capillary was hydrophilized with the treatment of oxygen plasma (100 W) for 120 s. The aqueous continuous phase (W) was prepared by mixing PVA (1% w/v) in distilled water. The disperse phase (0) was prepared by dissolving 50 mg of 75:25 PLGA and 0.5 mg of Nile red in 5 mL dichloromethane for 15 min, followed by filtration with 0.22 μm PTFE syringe filter. W and O phases were infused from the two ends of the square capillary through the outer coaxial region using two syringe pumps at flow rates of 115 μL/min and 20 μL/min respectively. The fluids were hydrodynamically flow focused through the nozzle of the round capillary resulting in the formation of the emulsion droplets. 6 cm ID glass wells were used for sample collection. Approximately 125 μL of O/W emulsions were dispersed directly into the glass well containing a pre-dispensed film (1.0 mm) of distilled water. Solvent evaporation was performed at room temperature (25°) for 1 hr. The particles were washed three times using distilled water, placed in a pre-weighed vial and dried in vacuum for 3 hr. The weight of the particles was measured after desiccation.

### Example 6. Administration of Solid Polymer Formulation for Extended Ocular Residence Time in Rabbit Eyes

PLGA particles loaded with red fluorescent dye (Nile red) were prepared using the procedure described in Example 5. The particles were coated with chitosan by suspending them in a chitosan coating solution (0.5% (w/v) chitosan, 0.5% (v/v) glacial acetic acid in water, pH 5.0). A flake composition was prepared by combining the coated microparticles, 5-10% (w/v), with polyvinyl alcohol (PV), 2% (w/v) in water, adjusted to pH 5.5 with glacial acetic acid. 30 μL of the microparticle/PVA suspension was dried to yield a solid formulation. The solid formulation was a flake having dimensions of around 10 mm×6 mm×0.1 mm. The microparticles constituted 95-99% (w/w) of the final flake formulation.

A single-application, one week study was conducted with four rabbits using the flake formulation. Images of rabbit eye taken before administration and after at different time points. Highest fluorescent signal was observed at 30 min and 1 hr post-instillation in the lacrimal caruncle, upper and lower fornix areas, and can still be observed up to 12 hr post-instillation as well as in the roots of upper and lower eyelashes up to 144 hr post-instillation as shown in Table 2. Microparticles were retained in the preocular area for up to one week, without any signs of ocular inflammation or other adverse effects. All eyes treated had a healthy conjunctiva, normal discharge, normal iris and cornea (total score=0 for all) as examined at all time points, except for one eye which showed evidence of mild hyperemia on the conjunctiva (score 1) at 192 hr time point post-installation.

Table 2 shows the preocular residence time of the microparticles in rabbit eyes. Data are expressed as number of fluorescence-positive areas. Rating 0 represents no fluorescence positivity in any of 4 eyes for the region-of-interest while rating 4 represents the detection of fluorescence in all 4 eyes.

TABLE 2

| Time | | Cornea | Lacrimal caruncle | Upper fornix | Lower fornix |
|---|---|---|---|---|---|
| 30 | min | 1 | 3 | 2 | 3 |
| 1 | hr | 0 | 4 | 4 | 2 |
| 6 | hr | 1 | 2 | 1 | 1 |
| 12 | hr | 0 | 1 | 0 | 2 |
| 24 | hr | 0 | 0 | 0 | 0 |
| 48 | hr | 0 | 0 | 1 | 0 |
| 72 | hr | 0 | 0 | 1 | 0 |
| 96 | hr | 0 | 0 | 1 | 0 |
| 120 | hr | 0 | 0 | 0 | 0 |
| 144 | hr | 0 | 0 | 1 | 0 |
| 168 | hr | 0 | 0 | 0 | 0 |
| 192 | hr | 0 | 0 | 0 | 0 |

### Example 7. Lowering of Intraocular Pressure with Single-Dose Latanoprost Microparticle Formulation in Ocular Normotensive Dogs

Beagles were divided into 3 groups based on the baseline intraocular pressure (IOP) value on Day 0 with no difference among 3 groups. 50 μL of microparticle suspension (7), containing 0.577% (w/w) latanoprost, or vehicle was topically administered to the right eye in each dog at time 0. 50 μL of XALATAN, containing 0.005% (w/w) latanoprost, was topically administered to the right eye in each dog once daily for 7 days (Day 0 to Day 6) after the first TOP measurement each day. IOPs were measured twice per day (at around 10:20 and 16:50) using a TONOVET tonometer (Icare Finland Oy) on Day 0, Day 1, Day 3, and Day 6. IOP changes were calculated as the difference from the time 0 values. The microparticle suspension significantly lowered TOP at 6 and 24 hours after the administration of one dose as compared to vehicle (FIG. 6). XALATAN significantly lowered TOP at all measurement points as compared to vehicle. The microparticle suspension was dosed only once (on Day 0), while XALATAN was dosed multiple times (once daily from Day 0 to Day 6).

### Example 8. Study of Atropine Release from Microparticle Suspension

Atropine-loaded microparticles were manufactured as described above, using PLGA 502H (PLGA 50:50, MW=7,000-17,000). The particle size was 15-20 μm, and the drug loading was 4.6% (28% encapsulation efficiency). The particles were suspended in 100 μL of chitosan solution (0.5% w/v) containing acetic acid (0.5% v/v) for 1 hr, washed three times with distilled water, and dried under vacuum. Dried microparticles (5 mg) were added to 1 mL of PBS buffer in a plasma-pretreated glass vial and shaken at 225 rpm at 37° C. PBS (phosphate buffered saline) solution (pH 7.4) was used as the release medium. At specific time points up to 7 days, the vial was centrifuged for 10 min at 3220×g and 100 μL of the medium was withdrawn twice. Fresh PBS buffer was added to the solution in order to keep the volume of the release medium constant. Release samples were prepared by mixing equal volumes of release medium and acetonitrile for HPLC analysis.

The total amount of encapsulated atropine base in the dried microparticles was also investigated. 5 mg of dried microparticles were added to 1 mL of acetonitrile and sonicated for 15 minutes in an ice bath. The sample was then diluted with the HPLC mobile phase for analysis.

HPLC analysis was carried out on Shimadzu HPLC LC-20 series with a PDA detector. The separation was performed on a C18 chromatography column (Ace C18, 5 μm, 4.6×250 mm) at 30° C. using water with 6 mM phosphoric acid and acetonitrile in the ratio of 50:50 (v/v) as the mobile phase. The flow rate of the mobile phase was set at 1 mL/min and the detector wavelength was set at 220 nm. Atropine base eluted from the column with a characteristic retention time of 2.3 min. Cumulative release (%) was calculated using the percentage of cumulative amount of atropine base (μg) released at each time point over the total encapsulated atropine base in the dried microparticles.

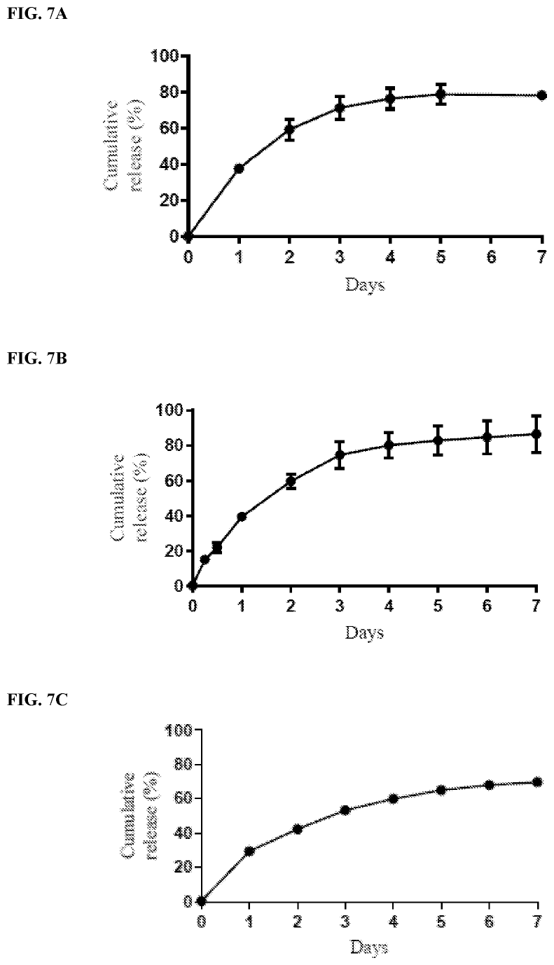

The total amount of encapsulated atropine base in the microparticles was 219±32 μg/mg, amounting to an encapsulation efficiency of 26±3% and drug loading of 4.4±0.6% (w/w). The release curve of atropine base from the microparticles is shown in FIG. 7A. More than 37% (w/w) of atropine was released within the first 24 hr, 59% (w/w) was released by 3 days, and subsequent slow release resulted in 78% (w/w) cumulative release at the end of 7 days. First-order release kinetics were observed, and the microparticle suspension was shown to provide sustained release of atropine base for up to 7 days.

### Example 9. Study of Brimonidine Release from Microparticle Suspension

Brimonidine-loaded microparticles were manufactured as described above, using PLGA 502H (PLGA 50:50, MW=7,000-17,000). The particle size was 15-20 μm, and the drug loading was 3.4% (50% encapsulation efficiency). The particles were coated with chitosan and combined with PBS for assay of drug release as described in Example 8. Release samples were prepared by mixing equal volumes of release medium and methanol for HPLC analysis. Samples representing the total amount of encapsulated brimonidine were also prepared using acetonitrile as described in Example 8. HPLC was performed using 10 mM phosphate buffer, pH=3, and methanol in a 50:50 (v/v) ratio as the mobile phase at a flow rate of 1 mL/min. The detector wavelength was set at 256 nm, and brimonidine base eluted with a characteristic retention time of 2.7 min.

The total amount of encapsulated brimonidine base in the microparticles was 34±2 μg/mg, amounting to an encapsulation efficiency of 54±3% and drug loading of 3.4±2% (w/w). The release curve of brimonidine base from the sample is shown in FIG. 7B. More than 40% (w/w) of brimonidine base was released within the first 24 hr, 20% (w/w) was released by 3 days, and subsequent slow release resulted in 80% (w/w) cumulative release at the end of 7 days. First-order release kinetics were observed, and the microparticle suspension was shown to provide sustained release of brimonidine base for up to 7 days.

### Example 10. Study of Timolol Release from Microparticle Suspension

Timolol-loaded microparticles were manufactured as described above, using PLGA 502H (PLGA 50:50, MW=7,000-17,000). The particle size was 15-20 μm, and the drug loading was 5.3% (32% encapsulation efficiency). The particles were coated with chitosan and combined with PBS for assay of drug release as described in Example 8. Release samples were prepared by mixing equal volumes of release medium and acetonitrile containing 0.1% acetic acid for HPLC analysis. Samples representing the total amount of encapsulated brimonidine were also prepared using acetonitrile as described in Example 8. HPLC was performed at 25° C., using water (+0.1% acetic acid) and acetonitrile (+0.1% acetic acid) in a 40:60 (v/v) ratio as the mobile phase at a flow rate of 1 mL/min. The detector wavelength was set at 295 nm, and timolol base eluted with a characteristic retention time of 2.0 min.

The total amount of encapsulated timolol base in the microparticles was 53 μg/mg, amounting to an encapsulation efficiency of 32% and drug loading of 5.3% (w/w). The release curve of timolol base from the microparticles is shown in FIG. 7C. More than 29% (w/w) of timolol base was released within the first 24 hr, 50% (w/w) was released by 3 days, and subsequent slow release resulted in 69% (w/w) cumulative release at the end of 7 days. First-order release kinetics were observed, and the microparticle suspension was shown to provide sustained release of timolol base for up to 7 days.

### Example 11. Study of Carbonic Anhydrase Inhibitor Release from Microparticle Suspensions

Brinzolamide- and dorzolamide-loaded microparticles were manufactured as described above, using PLGA 502H (PLGA 50:50, MW=7,000-17,000). The microparticles were suspended with 100 μL of 0.5% (w/v) chitosan solution with 0.5% (v/v) acetic acid for 1 hr, washed three times with distilled water, and dried under vacuum.

5 mg of dried microparticles was combined with 1 mL of PBS buffer in a plasma-pretreated glass vial and shaken at 225 rpm at 37° C. 1×PBS (phosphate buffered saline) solution (pH 7.4) was used as the release medium. At specific time points up to 7 days, the vial was centrifuged for 10 min at 3220×g and 100 μL of the medium was withdrawn twice. Fresh PBS buffer was added to the solution in order to keep the volume of the release medium constant. Release samples were prepared by mixing with equal volumes of acetonitrile for HPLC analysis.

The total amount of encapsulated drug from the dried microparticles was also investigated. 5 mg of dried microparticles was added to 1 mL of acetonitrile and sonicated for 15 minutes in an ice bath. Samples were then diluted with the HPLC mobile phase for analysis.

HPLC analysis was carried out on Agilent 1200 HPLC with a VWD detector. The separation was performed on a C18 chromatography column (Ace C18, 5 μm, 4.6×250 mm) at 25° C. using 50 mM phosphate buffer pH 6.6, acetonitrile, and methanol (45:15:40) as the mobile phase. The flow rate of the mobile phase was set at 1 mL/min and the detector wavelength was set at 254 nm. The retention times observed for brinzolamide and dorzolamide under these conditions were 3.0 min and 2.2 min, respectively. Cumulative release (%) was calculated using the percentage of cumulative amount of drug (μg) released at each time point over the total encapsulated drug in the dried microparticles.

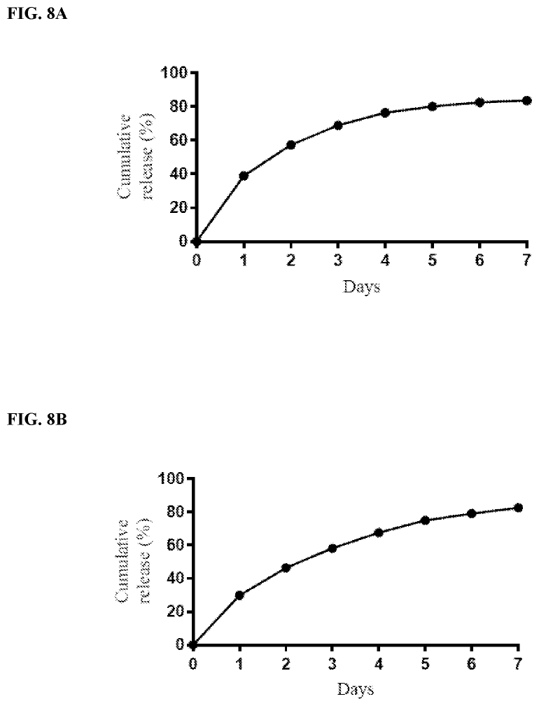

The total amount of encapsulated brinzolamide in the microparticles was 25 μg/mg, amounting to an encapsulation efficiency of 15% and drug loading of 2.6% (w/w). The total amount of encapsulated dorzolamide in the microparticles was 14 μg/mg, amounting to an encapsulation efficiency of 9% and drug loading of 1.4% (w/w). The release curves of brinzolamide and dorzolamide from the microparticles are shown in FIG. 8A and FIG. 8B, respectively. More than 38% (w/w) of brinzolamide was released within the first 24 hr, 68% (w/w) was released by 3 days, and subsequent slow release resulted in 83% (w/w) cumulative release at the end of 7 days. Similarly, dorzolamide was 29% (w/w) of dorzolamide was released within the first 24 hr, 58% (w/w) was released by 3 days, and 82% (w/w) was released by 7 days. First-order kinetics were observed in both cases. The microparticle suspensions were shown to provide sustained release of carbonic anhydrase inhibitors brinzolamide and dorzolamide for up to 7 days.

### Example 12. Study of UV-Blocking Agent Release from Microparticle Suspensions

Microparticles loaded with UV blocking agents octyl methoxycinnamate (OMC) and benzophenone-3 (BP3) were manufactured as described above, using PLGA 502H (PLGA 50:50, MW=7,000-17,000). The microparticles were suspended with 100 μL of 0.5% (w/v) chitosan solution with 0.5% (v/v) acetic acid for 1 hr, washed three times with distilled water, and dried under vacuum.

5 mg of dried microparticles was combined with 1 mL of PBS buffer in a plasma-pretreated glass vial and shaken at 225 rpm at 37° C. 1×PBS (phosphate buffered saline) solution (pH 7.4) with 1% Tween-80 was used as the release medium. At specific time points up to 7 days, the vial was centrifuged for 10 min at 3220×g and 100 μL of the medium was withdrawn twice. Fresh PBS buffer was added to the solution in order to keep the volume of the release medium constant. Release samples were prepared by mixing with equal volumes of acetonitrile for HPLC analysis.

The total amount of encapsulated UV-blocking agents from the dried microparticles was also investigated. 5 mg of dried microparticles were added to 1 mL of acetonitrile and sonicated for 15 minutes in an ice bath. Samples were then diluted with the HPLC mobile phase for analysis.

HPLC analysis was carried out on Agilent 1200 HPLC with a VWD detector. The separation was performed on a C18 chromatography column (Ace C18, 5 μm, 4.6×250 mm) at 25° C. using acetonitrile and water (88:12) as the mobile phase. The flow rate of the mobile phase was set at 1 mL/min and the detector wavelength was set at 310 nm for OMC and 287 nm for BP-3. The retention times observed for OMC and BP-3 under these conditions were 10.3 min and 4.2 min, respectively. Cumulative release (%) was calculated using the percentage of cumulative amount of UV-blocking agent (μg) released at each time point over the total amount encapsulated in the dried microparticles.

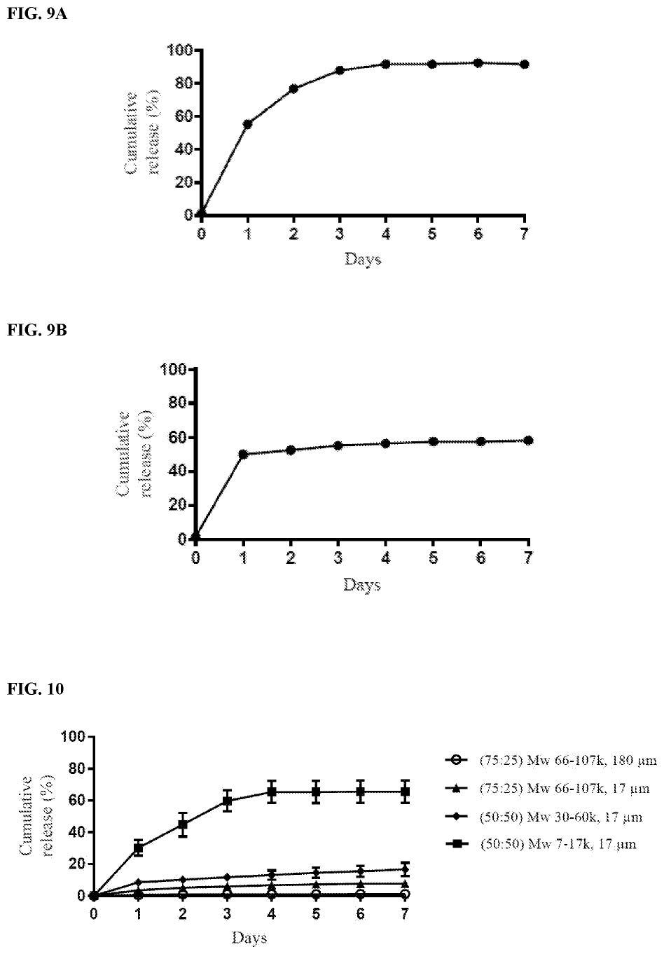

The total amount of encapsulated OMC in the microparticles was 205 μg/mg, amounting to an encapsulation efficiency of 100% and drug loading of 20% (w/w). The total amount of encapsulated BP-3 in the microparticles was 210 μg/mg, amounting to an encapsulation efficiency of 100% and drug loading of 21% (w/w). The release curves of OMC and BP-3 from the microparticles are shown in FIG. 9A and FIG. 9B, respectively. More than 55% (w/w) of OMC was released within the first 24 hr, 88% (w/w) was released by 3 days, and subsequent slow release resulted in 91% (w/w) cumulative release at the end of 7 days. Interestingly, most of the BP-3 release—49% (w/w)—occurred within the first 24 hr, while subsequent slow release resulted in 58% (w/w) cumulative release at the end of 7 days. The microparticle suspensions were shown to provide sustained release of UV-blocking agents OMC and BP-3 for up to 7 days.

### Example 13. Study of Drug Release from Microparticle Suspensions of Varying Size

Microparticle suspensions were compared to study the effect of particle size and polymer composition on drug release characteristics. The ratio of polymer to drug used in the loading procedure was 5:1 w/w.

Manufactured microparticles were suspended in 100 μL of 0.5% (w/v) chitosan solution containing 0.5% (v/v) acetic acid for 1 hr, washed three times with distilled water, and dried under vacuum. 1-5 mg of dried microparticles were then combined with 1 mL of PBS buffer in a plasma-pretreated glass vial and shaken at 225 rpm at 37° C. 1×PBS (pH 7.4) was used as the release medium. At specific time points up to 7 days, vials were centrifuged for 10 min at 3220 g and 100 μL of the medium were withdrawn twice. Fresh PBS buffer was added to the solution in order to keep the volume of the release medium constant. Release samples were prepared by mixing equal volumes of acetonitrile for HPLC analysis.

Total amount of encapsulated latanoprost from the dried microparticles was also investigated. Five milligrams of dried microparticles were added to 1 mL of acetonitrile and sonicated for 15 minutes in iced water. Sample was diluted with HPLC mobile phase for analysis.

HPLC analysis was carried out on an Agilent 1200 HPLC with VWD detector. The separation was performed on a C18 chromatography column (Zorbax Eclipse Plus C18, 5 4.6×150 mm) at 25° C. using acetonitrile and water (70:30) as the mobile phase. The flow rate of the mobile phase was set at 1 mL/min and the detector wavelength was set at 210 nm. A characteristic retention time of 3.0 min was observed for latanoprost under these conditions. Cumulative release (%) was calculated using the percentage of cumulative amount of latanoprost (μg) released at each time point over the total encapsulated latanoprost in dried microparticles.

A summary of the properties of the different populations of latanoprost-loaded PLGA microparticles is set forth in Table 3.

TABLE 3

| | 8-1 | | 8-2 | | 8-3 | | 8-4 | |
|---|---|---|---|---|---|---|---|---|
| Polymer | PLGA 75:25 | | PLGA 75:25 | | PLGA 50:50 | | PLGA 50:50 | |
| | Mw | | Mw | | Mw | | Mw | |
| | 66,000-107,000 | | 66,000-107,000 | | 30,000-60,000 | | 7,000-17,000 | |
| Particle Size | 180 | μm | 17 | μm | 17 | μm | 17 | μm |
| Mean latanoprost | 147 | μg/mg | 135 | μg/mg | 109 | μg/mg | 133 | μg/mg |
| encapsulated | | | | | | | | |

| | | | | |
|---|---|---|---|---|
| Mean | 87% | 81% | 65% | 80% |
| encapsulation | | | | |
| efficiency | | | | |
| Mean drug | 14% (w/w) | 13% (w/w) | 11% (w/w) | 13% (w/w) |
| loading | | | | |

The release curve of latanoprost from the four different formulations are shown in FIG. 10. The fastest releasing 8-4 microparticles reached more than 29% (w/w) of latanoprost within the first 24 hr and up to 65% (w/w) cumulative release at the end of 7 days. Formulations are ranked 8-4>8-3>8-1>8-2 based on the release rates. While all four formulations showed sustained release, formulation 8-4 exhibited an exceptionally high cumulative release for up to 7 days.

Although the foregoing invention has been described in some detail by way of illustration and example for purposes of clarity of understanding, one of skill in the art will appreciate that certain changes and modifications may be practiced within the scope of the appended claims. In addition, each reference provided herein is incorporated by reference in its entirety to the same extent as if each reference was individually incorporated by reference. Where a conflict exists between the instant application and a reference provided herein, the instant application shall dominate.

What is claimed is:

1. A composition comprising a population of polymer particles comprising a cargo, wherein the particles have an average particle size ranging from about 1 μm to about 25 μm.

2. The composition of claim 1, wherein the particles are adapted to carry and release the cargo upon topical ocular administration to a subject.

3. The composition of claim 1, wherein the polymer is selected from the group consisting of poly(lactic-co-glycolic acid), polylactic acid, poly(glycolic acid), poly(acrylic acid), alginate, a poly(alkyl cyanoacrylate), cellulose acetate phthalate, poly(ethyl cyanoacrylate), poly(hexadecyl cyanoacrylate), polycaprolactone, polylactic acid-polyethylene glycol copolymer, poly(lactic-co-glycolic acid)-polyethylene glycol copolymer, and combinations thereof.

4. The composition of claim 3, wherein the polymer is poly(lactic-co-glycolic acid).

5. The composition of claim 4, wherein the poly(lactic-co-glycolic acid) has a molecular weight ranging from about 4 kDa to about 150 kDa (weight average).

6. The composition of claim 4, wherein the molecular weight ranges from about 7 kDa to about 17 kDa (weight average).

7. The composition of claim 4, wherein the molar ratio of lactic acid to glycolic acid in the poly(lactic-co-glycolic acid) ranges from about 5:95 to about 95:5.

8. The composition of claim 7, wherein the molar ratio of lactic acid to glycolic acid is about 50:50.

9. The composition of claim 1, wherein the average particle size ranges from about 10 μm to about 20 μm.

10. The composition of claim 1, wherein the cargo comprises one or more ophthalmic therapeutic agents.

11. The composition of claim 10, wherein the cargo comprises two or more ophthalmic therapeutic agents.

12. The composition of claim 1, wherein the cargo comprises a prostaglandin, a carbonic anhydrase inhibitor, an alpha agonist, a beta blocker, a UV blocker, or a combination thereof.

13. The composition of claim 1, wherein the cargo comprises latanoprost.

14. The composition of claim 1, wherein the amount of cargo ranges from about 0.1% (w/w) to about 50% (w/w) based on the total weight of the particles.

15. The composition of claim 14, wherein the amount of cargo ranges from 1% (w/w) to about 20% (w/w) based on the total weight of the particles.

16. The composition of claim 1, wherein the cargo comprises a further population of particles, the further population of particles comprising at least one drug.

17. The composition of claim 1, wherein the particles are coated with a mucoadhesive coating.

18. The composition of claim 17, wherein the mucoadhesive coating comprises chitosan.

19. The composition of claim 1, wherein:
the average particle size ranges from about 10 μm to about 20 μm;
the polymer is poly(lactic-co-glycolic acid) having a molecular weight ranging from about 7 kDa to about 17 kDa (weight average), wherein the molar ratio of lactic acid to glycolic acid in the poly(lactic-co-glycolic acid) is about 50:50;
the cargo comprises a prostaglandin, a carbonic anhydrase inhibitor, an alpha agonist, a beta blocker, a UV blocker, or a combination thereof;
the amount of cargo ranges from 1% (w/w) to about 20% (w/w) based on the total weight of the particles; and
the particles are coated with a mucoadhesive polymer comprising chitosan.

20. The composition of claim 1, wherein the particles are suspended in a fluid medium.

21. The composition of claim 1, wherein the particles are partially or fully embedded in a solid polymer matrix.

22. The composition of claim 21, wherein the solid polymer matrix comprises one or more polymers selected from the group consisting of polyvinyl alcohol, poly(ethylene glycol), polyvinyl pyrrolidone, polyacrylic acid, polyacrylamide, poly(N-2-hydroxypropyl) methylacrylamide), poly(methyl vinyl ether-alt-maleic anhydride), and a poly(2-alkyl-2-oxazoline).

23. The composition of claim 21, wherein the solid polymer matrix comprises polyvinyl alcohol.

24. The composition of claim 1, which is formulated as an ophthalmic composition for administration to an eye of the subject.

25. A composition according to any one of claims 1-24 for use in the treatment of an ocular disease or disorder in a patient.

26. The composition of claim 25, wherein the ocular disease or disorder is glaucoma.

27. A composition according to any one of claims 1-24 for use in the manufacture of a medicament for the treatment of an ocular disease or disorder.

28. The composition of claim 27, wherein the ocular disease and/or disorder is glaucoma.

29. A method for treating glaucoma, the method comprising administering an effective amount of a composition according to any one of claims 1-24 to a subject in need thereof.

30. A kit comprising a first container comprising a composition according to any one of claims 1-20 and a second container comprising a fluid medium for suspension of the particles in the composition, wherein the fluid medium optionally comprises one or more pharmaceutically acceptable excipients.

31. The kit of claim 30, wherein the fluid medium is aqueous.

32. The kit of claim 30 or claim 31, wherein the fluid medium comprises dissolved cargo.

33. The kit of any one of claims 30-32, wherein the concentration of the dissolved cargo is at or around the solubility limit of the cargo in the fluid medium.

34. The kit of claim 30, further comprising instructions for suspending the particles in the fluid medium.
